(19) United States
(12) Patent Application Publication
(10) Pub. No.: US 2021/0186481 A1
(43) Pub. Date: Jun. 24, 2021

(54) PRE-SHAPED TISSUE ANCHORS AND NEEDLES FOR TISSUE ANCHOR DEPLOYMENT

(71) Applicant: Edwards Lifesciences Corporation, Irvine, CA (US)

(72) Inventors: John Richard Carpenter, Santa Ana, CA (US); Louis A. Campbell, Santa Ana, CA (US)

(21) Appl. No.: 17/191,613

(22) Filed: Mar. 3, 2021

Related U.S. Application Data
(63) Continuation of application No. PCT/US2019/049356, filed on Sep. 3, 2019.
(60) Provisional application No. 62/838,438, filed on Apr. 25, 2019.
(60) Provisional application No. 62/727,628, filed on Sep. 6, 2018.

(51) Int. Cl. A61B 17/04; A61B 17/06

(52) U.S. Cl. CPC A61B 17/0401; A61B 2017/0409; A61B 2017/0412; A61B 17/06066

(57) ABSTRACT

A tissue anchor includes a memory metal wire configured to transition between an at least partially straightened delivery configuration and an expanded deployed configuration forming an anchor and a suture-attachment feature configured to have a suture coupled thereto. In the expanded deployed configuration, one or more portions of the memory metal wire extend radially outward from a center of the tissue anchor.

### RELATED APPLICATIONS

This application is a continuation of International Patent Application No. PCT/US2019/049356, filed Sep. 3, 2019, which claims the benefit of U.S. Patent Application No. 62/727,628, filed Sep. 6, 2018, and of U.S. Patent Application No. 62/838,438, filed Apr. 25, 2019, the entire disclosures of which are incorporated by reference for all purposes.

### BACKGROUND

### Technical Field

The disclosure herein relates to devices for anchoring to biological tissue.

### Description of Related Art

Biocompatible implant devices, such as heart valves, may be implanted in patients to treat various conditions. Anchoring to cardiac tissue can be associated with certain complications and/or issues.

### SUMMARY

In some implementations, the present disclosure relates to a tissue anchor comprising a memory metal wire configured to transition between an at least partially straightened delivery configuration and an expanded deployed configuration forming an anchor, and a suture-attachment feature configured to have a suture coupled thereto. In the expanded deployed configuration, one or more portions of the memory metal wire extend radially outward from a center of the tissue anchor.

In the expanded deployed configuration, the memory metal wire may form a plurality of loop projections. For example, the suture-attachment feature may comprise a connection portion of the memory metal wire between circumferentially-spaced radial inner ends of adjacent loop projections of the plurality of loop projections. In some embodiments, the tissue anchor further comprises a suture coupled to the suture-attachment feature and having one or two suture tails extending therefrom.

In the expanded deployed configuration, the memory metal wire may form a clover form. For example, the clover form may have two free ends. In some embodiments, the memory metal wire is flat in the expanded deployed configuration. In some embodiments, the memory metal wire is configured to transition to the expanded deployed configuration in response to a stimulus. In the expanded deployed configuration, the memory metal wire may form a spiral form or a grill form.

In some implementations, the present disclosure relates to an anchor delivery system comprising a main shaft having an atraumatic tip and an interior lumen, a needle having a distal end and an interior lumen, the needle being disposed within the interior lumen of the main shaft and configured to be extended from the distal end of the main shaft in a deployed position of the needle, a pusher disposed within the interior lumen of the needle and configured to be extended from the distal end of the needle in a deployed position of the pusher. The anchor delivery system further comprises a memory metal wire disposed in the interior lumen of the needle in an at least partially straightened delivery configuration, the memory metal wire being configured to automatically assume an expanded deployed configuration when ejected from the interior lumen of the needle by the pusher, and a suture coupled to a suture-attachment feature of the memory metal wire within the interior lumen of the needle.

In some implementations, the present disclosure relates to a method of deploying a tissue anchor. The method comprises providing an anchor delivery system comprising a main shaft having an atraumatic tip and an interior lumen, a needle having a distal end and an interior lumen, the needle being disposed within the interior lumen of the main shaft and configured to be extended from the distal end of the main shaft in a deployed position of the needle, a pusher disposed within the interior lumen of the needle and configured to be extended from the distal end of the needle in a deployed position of the pusher, a memory metal wire disposed in the interior lumen of the needle in an at least partially straightened delivery configuration, the memory metal wire being configured to automatically assume an expanded deployed configuration when ejected from the interior lumen of the needle by the pusher, and a suture coupled to a suture-attachment feature of the memory metal wire within the interior lumen of the needle. The method further comprises positioning the atraumatic tip of the main shaft against a target tissue, moving the needle to the deployed position, thereby puncturing through the target tissue with the needle, ejecting the memory metal wire from the interior lumen of the needle while the needle is in the deployed position, and forming a memory metal wire into an expanded tissue anchor form on a distal side of the target tissue.

In some embodiments, the method further comprises pre-shaping the memory metal wire in the expanded tissue anchor form, compressing the memory metal wire into a compressed delivery configuration, and inserting the memory metal wire into the interior lumen of the needle in the compressed delivery configuration. In some embodiments, the expanded tissue anchor form has a clover shape comprising a plurality of radially-extending loop projections. Moving the needle to the deployed position may comprise puncturing the target tissue with a point of the needle being substantially aligned with a longitudinal axis of the main shaft. For example, the needle may comprise an elongated shaft forming the interior lumen of the needle, the elongated shaft having a bend feature that is configured to align the point of the needle with a longitudinal axis of the elongated shaft.

In some implementations, the present disclosure relates to a needle comprising a tip portion comprising a sharp point one or more distal beveled surfaces, and a proximal beveled surface. The needle further comprises an elongated shaft forming an interior lumen. The elongated shaft includes a bend configured to align the sharp point of the needle with a longitudinal axis of the elongated shaft.

The proximal beveled surface and at least a portion of the one or more distal beveled surfaces may be radiused surfaces. For example, the radiused surfaces can be formed using electropolishing. In some embodiments, portions of the one or more distal beveled surfaces adjacent to the point of the needle are not radiused. In some embodiments, the bend has an angle between about 3°-5°.

In some implementations, the present disclosure relates to a needle delivery assembly comprising a main shaft, having a distal end and an interior lumen, a needle having a distal end and an interior lumen, wherein the needle is configured to be slidably disposed (e.g., slip-fit) within the interior lumen of the main shaft in a stored position of the needle, and to extend from the distal end of the main shaft in a deployed position of the needle, an ejector configured to be slidably disposed within the interior lumen of the needle in a stored position of the ejector, and to extend from the distal end of the needle in a deployed position of the ejector, a repair device configured to be slidably disposed in the needle, and a suture connected to the repair device. The ejector is configured to push the repair device at least partially out of the needle when the ejector is moved from the stored position of the ejector to the deployed position of the ejector.

In some embodiments, the ejector comprises an interior lumen, the suture is disposed at least partially within the interior lumen of the ejector, and the interior lumen of the ejector is sized to prevent the repair device from entering into the interior lumen of the ejector. The interior lumen of the main shaft can be sized to accommodate a second needle slidably disposed therein. In some embodiments, the distal end of the needle comprises a tip, the tip is disposed against a wall of the interior lumen of the main shaft in the stored position of the needle, and the tip is positioned near a center of the interior lumen of the main shaft in the deployed position of the needle. The distal end of the main shaft can comprise an atraumatic blunt end, an expandable balloon, and/or a suction device.

In some implementations, the present disclosure relates to a needle comprising a distal end and an interior lumen. The distal end comprises a tip, a distal beveled edge, and a proximal beveled edge. The proximal beveled edge and at least part of the distal beveled edge have a radiused surface. In some embodiments, an entirety of the distal beveled edge is radiused. The radiused surface can be electropolished. A radius of the radiused surface may be between about 25 and about 500 μm (about 0.001 and about 0.02 inches), and/or between about 130 and about 400 μm (about 0.005 and about 0.015 inches). The tip can be electropolished. A radius of the tip can be between about 25 and about 250 μm (about 0.001 and about 0.01 inches). In some embodiments, the radius of the tip is between about 25 and about 130 μm (about 0.001 and about 0.005 inches).

In some implementations, the present disclosure relates to a needle comprising a distal end and an interior lumen. The distal end comprises a tip and is angled such that the tip is aligned with a central axis of the needle.

In some implementations, the present disclosure relates to a needle comprising a distal end and an interior lumen. The distal end comprises a tip, and the tip is coincident with the interior lumen of the needle.

In some implementations, the present disclosure relates to a needle comprising a distal end and an interior lumen. The distal end comprises a tip. An ejector is slidably disposed (e.g., slip-fit) within the interior lumen. A position of the tip is adjacent to an outside surface of the ejector.

In some implementations, the present disclosure relates to a repair method comprising providing a needle delivery assembly. The needle delivery assembly comprises a main shaft having a distal end and an interior lumen. The needle delivery assembly further comprises a needle having a distal end and an interior lumen, wherein the needle is configured to be slidably disposed (e.g., slip-fit) within the interior lumen of the main shaft in a stored position of the needle, and to extend from the distal end of the main shaft in a deployed position of the needle. The needle delivery assembly further comprises an ejector configured to be slidably disposed within the interior lumen of the needle in a stored position of the ejector, and to extend from the distal end of the needle in a deployed position of the ejector. The needle delivery assembly further comprises a repair device configured to be slidably disposed in the interior lumen of the needle, and a suture connected to the repair device. The method comprises positioning the distal end of the main shaft at a target tissue, puncturing through the target tissue with the needle, advancing and pushing, using the ejector, the repair device out of the interior lumen of the needle while the needle is in a puncture position, and withdrawing the main shaft, the needle, and the ejector from the target tissue.

In some implementations, the present disclosure relates to a repair method comprising providing a needle delivery assembly. The needle delivery assembly comprises a main shaft having a distal end and an interior lumen, and a first needle having a distal end and an interior lumen, wherein the first needle is slidably disposed (e.g., slip-fit) within the interior lumen of the main shaft in a stored position, the distal end of the first needle extends from the distal end of the main shaft in a deployed position. The needle delivery assembly further comprises a first ejector slidably disposed within the interior lumen of the first needle in a stored position, the distal end of the first ejector extends from the distal end of the first needle in a deployed position. The needle delivery assembly further comprises a first repair device slidably disposed in the first needle, and a first suture connected to the first repair device. The needle delivery assembly further comprises a second needle having a distal end and an interior lumen, wherein the second needle is slidably disposed within the interior lumen of the main shaft in a stored position, the distal end of the second needle extends from the distal end of the main shaft in a deployed position. The needle delivery assembly further comprises a second ejector slidably disposed within the interior lumen of the second needle in a stored position, the distal end of the second ejector extends from the distal end of the second needle in a deployed position. The needle delivery assembly further comprises a second repair device slidably disposed in the second needle, and a second suture connected to the second repair device. The first ejector is configured to push the first repair device out of the first needle when the first ejector extends from the distal end of the first needle in the deployed position. The second ejector is configured to push the second repair device out of the second needle when the second ejector extends from the distal end of the second needle in the deployed position. The method further comprises positioning the distal end of the main shaft at a first target tissue area, positioning a tip of the first needle near a center of the main shaft, puncturing, using the first needle, through the first target tissue area, advancing and pushing, using the first ejector, the first repair device out of the first needle while the first needle is adjacent to the first target tissue, and withdrawing the main shaft, the first needle, and the first ejector from the first target tissue.

In some embodiments, the method further comprises positioning the distal end of the main shaft at a second target tissue, positioning a tip of the second needle near the center of the main shaft, puncturing, by the second needle, through the second target tissue, advancing and pushing, by the second ejector, the second repair device out of the second needle while the second needle is adjacent to the second target tissue, and withdrawing the main shaft, the second needle, and the second ejector from the tissue.

### BRIEF DESCRIPTION OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 is a cut-away anterior view of the human heart showing the internal chambers, valves, and adjacent structures.

FIG. 2 is a perspective view of a healthy mitral valve with the leaflets closed.

FIG. 3 is a top view of a dysfunctional mitral valve with a visible gap between the leaflets.

FIG. 4 shows a simplified cross-sectional view of a heart with four chambers and an apex region.

FIG. 5 illustrates the advancement of a device through an accessed region of the heart.

FIG. 6 shows an exemplary embodiment of a needle penetrating through a valve leaflet.

FIG. 7 shows an exemplary embodiment of a needle penetrating through a valve annulus.

FIG. 8 is a cross-sectional view of an exemplary embodiment of a needle delivery device.

FIG. 9 illustrates the needle delivery device shown in FIG. 8 with the needle penetrating the tissue.

FIG. 10 illustrates the needle delivery device shown in FIG. 8 with the pledget deployed by the pledget ejector.

FIG. 11 illustrates the needle delivery device shown in FIG. 8 with the pledget deployed in place and the needle delivery device retracting.

FIG. 12A is a side view of an exemplary embodiment of a needle.

FIG. 12B is a view taken in the direction of arrows 12B-12B in FIG. 12A.

FIG. 13 is a side view of an exemplary embodiment of a needle.

FIG. 14 is a side view of an exemplary embodiment of a needle.

FIG. 15 is a side view of an exemplary embodiment of a needle.

FIG. 16 shows the needle of FIG. 15 with a pusher extending from an end of the needle.

FIG. 17 is a perspective, partially sectioned view of an exemplary embodiment of a needle delivery device with four needles.

FIG. 18 illustrates the needle delivery device shown in FIG. 17 with one of the needles optionally rotating and extending to penetrate the tissue.

FIG. 19 illustrates the needle delivery device shown in FIG. 17 with a pledget deployed from the extended needle shown in FIG. 18.

FIG. 20 illustrates the needle delivery device shown in FIG. 17 with the pledget deployed in place and the needle delivery device retracting from the tissue.

FIG. 21 illustrates the needle delivery device shown in FIG. 17 with the needle and the pledget ejector retracting into the main shaft.

FIG. 22 illustrates the needle delivery device shown in FIG. 17 with the needle and pledget ejector back to the main shaft.

FIG. 23A-23C illustrate an exemplary embodiment of an anchor member.

FIGS. 24A-24C illustrates an exemplary procedure for securing an exemplary embodiment of an attachment member to a tissue member with the exemplary anchor member of FIGS. 23A-23C.

FIGS. 25-26 illustrate another exemplary embodiment of an anchor member.

FIG. 27 illustrates a needle having a deflected tip in accordance with one or more embodiments.

FIGS. 28A-28C illustrate views of a pre-shaped tissue anchor in accordance with one or more embodiments.

FIGS. 29-31 illustrate stages of a procedure for deploying a pre-shaped tissue anchor using a tissue anchor delivery system in accordance with one or more embodiments.

FIGS. 32-34 illustrate additional example tissue anchor forms in accordance with embodiments.

### DETAILED DESCRIPTION

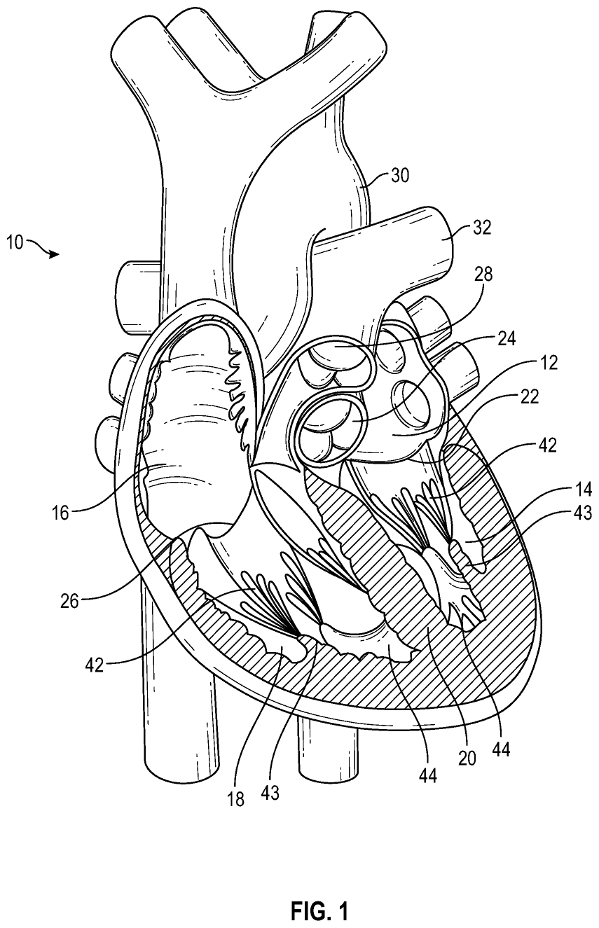

As illustrated in FIG. 1, the human heart 10 has four chambers, which include two upper chambers denoted as atria 12, 16 and two lower chambers denoted as ventricles 14, 18. A septum 20 divides the heart 10 and separates the left atrium 12 and left ventricle 14 from the right atrium 16 and right ventricle 18. The heart further contains four valves 22, 24, 26, and 28. The valves function to maintain the pressure and unidirectional flow of blood through the body and to prevent blood from leaking back into a chamber from which it has been pumped.

Two valves separate the atria 12, 16 from the ventricles 14, 18, denoted as atrioventricular valves. The left atrioventricular valve, the mitral valve 22, controls the passage of oxygenated blood from the left atrium 12 to the left ventricle 14. A second left valve, the aortic valve 24, separates the left ventricle 14 from the aortic artery (aorta) 30, which delivers oxygenated blood via the circulation to the entire body. The aortic valve 24 and mitral valve 22 are part of the “left” heart, which controls the flow of oxygen-rich blood from the lungs to the body. The right atrioventricular valve, the tricuspid valve 26, controls passage of deoxygenated blood into the right ventricle 18. A fourth valve, the pulmonary valve 28, separates the right ventricle 18 from the pulmonary artery 32. The right ventricle 18 pumps deoxygenated blood through the pulmonary artery 32 to the lungs wherein the blood is oxygenated and then delivered to the left atrium 12 via the pulmonary vein. Accordingly, the tricuspid valve 26 and pulmonic valve 28 are part of the “right” heart, which control the flow of oxygen-depleted blood from the body to the lungs.

Both the left and right ventricles 14, 18 constitute “pumping” chambers. The aortic valve 24 and pulmonic valve 28 lie between a pumping chamber (ventricle) and a major artery or vein and control the flow of blood out of the ventricles and into circulation. The aortic valve 24 and pulmonic valve 28 normally have three cusps, or leaflets, that open and close and thereby function to prevent blood from leaking back into the ventricles after being ejected into the lungs or aorta 30 for circulation.

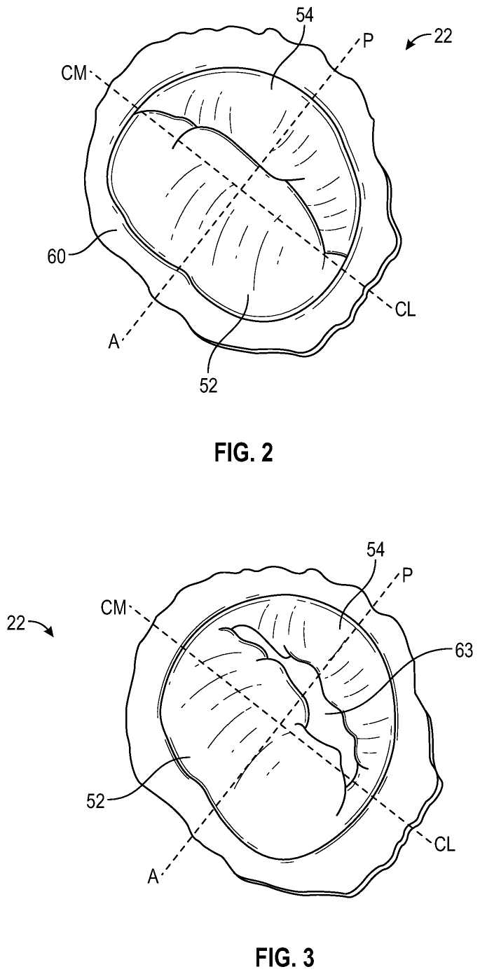

Both the left and right atria 12, 16 are “receiving” chambers. The mitral valve 22 and tricuspid valve 26, therefore, lie between a receiving chamber (atrium) and a ventricle so as to control the flow of blood from the atria to the ventricles and prevent blood from leaking back into the atrium during ejection into the ventricle. The mitral valve 22, includes two cusps, or leaflets (shown in FIG. 2), and the tricuspid valve 26 normally include three cusps, or leaflets. The mitral valve 22 and the tricuspid valve 26 are encircled by a variably dense fibrous ring of tissues known as the annulus. The valves 22, 26 are anchored to the walls of the ventricles by chordae tendineae (chordae) 42. The chordae tendineae 42 are cord-like tendons that connect the papillary muscles 43 to the leaflets of the mitral valve 22 and tricuspid valve 26 of the heart 10. The papillary muscles 43 are located at the base of the chordae 42 and are within the walls of the ventricles. They serve to limit the movements of the mitral valve 22 and tricuspid valve 26 and prevent them from being reverted. The papillary muscles 43 do not open or close the valves of the heart, which close passively in response to pressure gradients; rather, the papillary muscles 43 brace the valve leaflets against the high pressure needed to circulate the blood throughout the body. Together, the papillary muscles 43 and the chordae tendineae 42 are known as the subvalvular apparatus. The function of the subvalvular apparatus is to keep the valves from prolapsing into the atria when they close.

As illustrated with reference to FIG. 2, the mitral valve 22 includes two leaflets, the anterior leaflet 52 and the posterior leaflet 54, and a diaphanous incomplete ring around the valve, called the annulus 60. The mitral valve 22 has two primary papillary muscles 43, the anteromedial and the posterolateral papillary muscles, which attach the leaflets 52, 54 to the walls of the left ventricle 14 via the chordae tendineae 42. The tricuspid valve 26 typically is made up of three leaflets with three papillary muscles. However, the number of leaflets can range between two and four. The three leaflets of the tricuspid valve 26 are referred to as the anterior, posterior, and septal leaflets. Although both the aortic and pulmonary valves each have three leaflets (or cusps), they do not have chordae tendineae.

Various disease processes can impair the proper functioning of one or more of the valves of the heart. These disease processes include degenerative processes (e.g., Barlow's disease, fibroelastic deficiency), inflammatory processes (e.g., rheumatic heart disease), and infectious processes (e.g., endocarditis). Additionally, damage to the ventricle from prior heart attacks (e.g., myocardial infarction secondary to coronary artery disease) or other heart diseases (e.g., cardiomyopathy) can distort the valve's geometry causing it to dysfunction. However, the vast majority of patients undergoing valve surgery, such as mitral valve surgery, suffer from a degenerative disease that causes a malfunction in a leaflet of the valve, which results in prolapse and regurgitation.

Generally, a heart valve can malfunction two different ways. One possible malfunction, valve steno sis, occurs when a valve does not open completely and thereby causes an obstruction of blood flow. Typically, stenosis results from buildup of calcified material on the leaflets of the valves causing them to thicken and thereby impairing their ability to fully open and permit adequate forward blood flow.

Another possible malfunction, valve regurgitation, occurs when the leaflets of the valve do not close completely thereby causing blood to leak back into the prior chamber. There are three mechanisms by which a valve becomes regurgitant or incompetent; they include Carpentier's type I, type II and type III malfunctions. A Carpentier type I malfunction involves the dilation of the annulus such that normally functioning leaflets are separated from each other and fail to form a tight seal (e.g., do not coapt properly). Included in a type I mechanism malfunction are perforations of the valve leaflets, as in endocarditis. A Carpentier's type II malfunction involves prolapse of one or both leaflets above the plane of coaptation. This is the most common cause of mitral regurgitation and is often caused by the stretching or rupturing of chordae tendineae normally connected to the leaflet. A Carpentier's type III malfunction involves restriction of the motion of one or more leaflets such that the leaflets are abnormally constrained below the level of the plane of the annulus. Leaflet restriction can be caused by rheumatic disease (IIIa) or dilation of the ventricle (IIIb).

FIG. 3 illustrates a mitral valve 22 having leaflets that do not properly coapt due to tethering and/or prolapse. Prolapse occurs when a leaflet 52, 54 of the mitral valve 22 is displaced into the left atrium 12 (see FIG. 1) during systole. Because one or more of the leaflets 52, 54 prolapse, the mitral valve 22 does not close properly, and, therefore, the leaflets fail to coapt. This failure to coapt causes a gap 63 between the leaflets 52, 54 that allows blood to flow back into the left atrium 12, during systole, while it is being ejected into the left ventricle 14. As set forth above, there are several different ways a leaflet can malfunction, which can thereby lead to regurgitation.

Although stenosis or regurgitation can affect any valve, stenosis is predominantly found to affect either the aortic valve 24 or the pulmonic valve 28, whereas regurgitation predominately affects either the mitral valve 22 or the tricuspid valve 26. Both valve stenosis and valve regurgitation increase the workload on the heart 10 and can lead to very serious conditions if left un-treated. Since the left heart is primarily responsible for circulating the flow of blood throughout the body, malfunction of the mitral valve 22 is particularly problematic and often life threatening. Accordingly, because of the substantially higher pressures on the left side of the heart, left-sided valve dysfunction is much more problematic.

Malfunctioning valves can either be repaired or replaced. Repair typically involves the preservation and correction of the patient's own valve. Replacement typically involves replacing the patient's malfunctioning valve with a biological or mechanical substitute. Typically, the aortic valve 24 and pulmonic valve 28 are more prone to stenosis. Because stenotic damage sustained by the leaflets is irreversible, the most conventional treatment for stenotic aortic and pulmonic valves is removal and replacement of the diseased valve. The mitral valve 22 and tricuspid valve 26, on the other hand, are more prone to deformation. Deformation of the leaflets, as described above, prevents the valves from closing properly and allows for regurgitation or back flow from the ventricle into the atrium, which results in valvular insufficiency. Deformations in the structure or shape of the mitral valve 22 or tricuspid valve 26 are often repairable.

An improperly functioning mitral valve 22 or tricuspid valve 26 is often repaired, rather than replaced. Conventional techniques for repairing a cardiac valve are labor-intensive, technically challenging, and require a great deal of hand-to-eye coordination. They can be, therefore, very challenging to perform, and require a great deal of experience and extremely good judgment. For instance, the procedures for repairing regurgitating leaflets can require resection of the prolapsed segment and insertion of an annuloplasty ring so as to reform the annulus of the valve. Additionally, leaflet sparing procedures for correcting regurgitation can be similarly labor-intensive and technically challenging, if not requiring an even greater level of hand-to-eye coordination. These procedures can involve the implantation of sutures (e.g., ePTFE suture, for example, GORE-TEX® sutures, W. L. Gore, Newark, Del.) so as to form artificial chordae in the valve. In these procedures, rather than performing a resection of the leaflets and/or implanting an annuloplasty ring into the patient's valve, the prolapsed segment of the leaflet is re-suspended using artificial chord sutures.

Regardless of whether a replacement or repair procedure is being performed, conventional approaches for replacing or repairing cardiac valves are typically invasive open-heart surgical procedures, such as sternotomy or thoracotomy, that require opening up of the thoracic cavity so as to gain access to the heart. Once the chest has been opened, the heart is bypassed and stopped. Cardiopulmonary bypass is typically established by inserting cannulae into the superior and inferior vena cavae (for venous drainage) and the ascending aorta (for arterial perfusion), and connecting the cannulae to a heart-lung machine, which functions to oxygenate the venous blood and pump it into the arterial circulation, thereby bypassing the heart. Once cardiopulmonary bypass has been achieved, cardiac standstill is established by clamping the aorta and delivering a “cardioplegia” solution into the aortic root and then into the coronary circulation, which stops the heart from beating. Once cardiac standstill has been achieved, the surgical procedure can be performed.

Needle delivery devices described by the present disclosure can be used in a wide variety of applications. In accordance with some embodiments disclosed herein, the heart can be accessed through one or more openings made by a relatively small incision(s) in a portion of the body proximal to the thoracic cavity, for instance, in between one or more of the ribs of the rib cage, proximate to the xyphoid appendage, or via the abdomen and diaphragm. Access to the thoracic cavity can be sought so as to allow the insertion and use of one or more thorascopic instruments, while access to the abdomen can be sought so as to allow the insertion and use of one or more laparoscopic instruments. Insertion of one or more visualizing instruments can then be followed by transdiaphragmatic access to the heart. Additionally, access to the heart can be gained by direct puncture (e.g., via an appropriately sized needle, for instance an 18-gauge needle) of the heart from the xyphoid region. Access can also be achieved using percutaneous means. Accordingly, the one or more incisions should be made in such a manner as to provide an appropriate surgical field and access site to the heart. See, e.g., “Full-Spectrum Cardiac Surgery Through a Minimal Incision Mini-Sternotomy (Lower Half) Technique”, Doty et al., Annals of Thoracic Surgery 1998; 65(2): 573-577 and “Transxiphoid Approach Without Median Sternotomy for the Repair of Atrial Septal Defects”, Barbero-Marcial et al., Annals of Thoracic Surgery 1998; 65(3): 771-774, which are specifically incorporated in their entirety herein by reference.

The term “minimally invasive” is used herein according to its broad and ordinary meaning and may refer to any manner by which an interior organ or tissue can be accessed with as little as possible damage being done to the anatomical structure through which entry is sought. Typically, a minimally invasive procedure is one that involves accessing a body cavity by a small incision made in the skin of the body. The term “small incision” is used according to its broad and ordinary meaning and may refer to an incision having a length generally of about 1 cm to about 10 cm, or about 4 cm to about 8 cm, or about 7 cm in length. The incision can be vertical, horizontal, or slightly curved. If the incision is placed along one or more ribs, the incision may follow the outline of the rib. The opening should extend deep enough to allow access to the thoracic cavity between the ribs or under the sternum and is preferably set close to the rib cage and/or diaphragm, dependent on the entry point chosen.

One or more other incisions can be made proximate to the thoracic cavity to accommodate insertion of a surgical scope. Such an incision is typically about 1 cm to about 10 cm, or about 3 cm to about 7 cm, or about 5 cm in length and should be placed near the pericardium so as to allow ready access to, and visualization of, the heart. The surgical scope can be any type of endoscope, a thorascope or laparoscope, depending upon the type of access and scope to be used. The scope may generally have a flexible housing and at least an about 16-times magnification. Insertion of the scope through an incision can allow a practitioner to analyze and “inventory” the thoracic cavity and the heart so as to further determine the clinical status of the subject and plan the procedure. For example, a visual inspection of the thoracic cavity can reveal important functional and physical characteristics of the heart and can indicate the access space (and volume) required at the surgical site and in the surgical field in order to perform the reparative cardiac valve procedure. At this point, the practitioner can confirm that access of one or more cardiac valves through the apex of the heart or another access site is appropriate for the particular procedure to be performed.

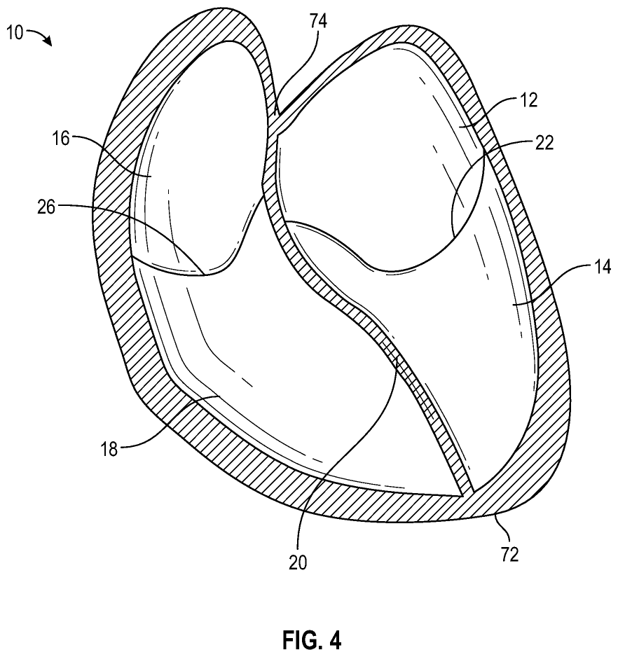

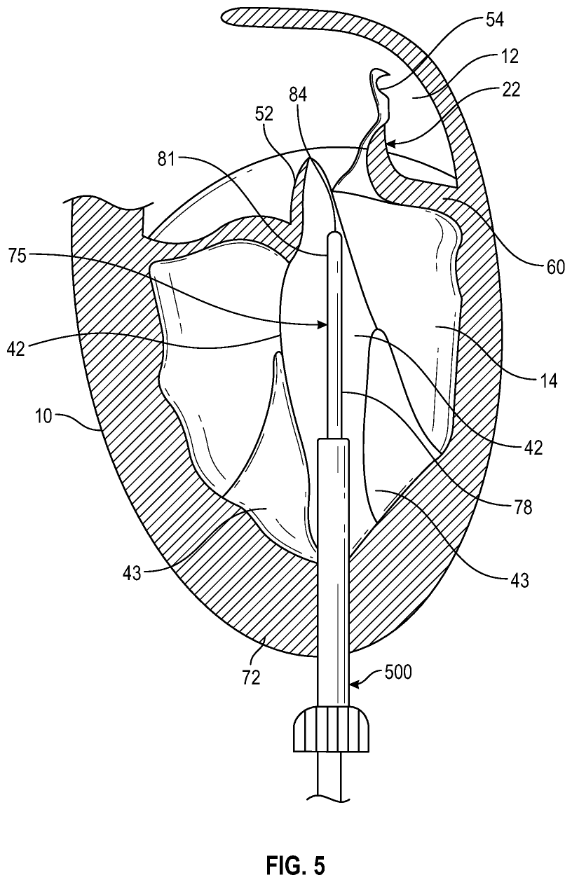

With reference to FIGS. 4 and 5, once a suitable entry point has been established, a suitable access device 500 (FIG. 5) can be advanced into the body in a manner so as to make contact with the heart 10. In some embodiments, the shaft/needle used for leaflet puncture is small enough and/or designed to penetrate into the thoracic cavity percutaneously, such that the access device 500 is not necessary. The advancement of the device can be performed in conjunction with sonography or direct visualization (e.g., direct transblood visualization). For instance, the device can be advanced in conjunction with TEE guidance or ICE so as to facilitate and direct the movement and proper positioning of the device for contacting the appropriate apical region of the heart. Typical procedures for use of echo guidance are set forth in Suematsu, Y., J. Thorac. Cardiovasc. Surg., 2005; 130:1348-1356, herein incorporated by reference in its entirety. However, the device 500 may be advanced in any manner.

Referring to FIGS. 4 and 5, one or more chambers 12, 14, 16, 18 in the heart 10 can be accessed by the device 500. Access into a chamber in the heart can be made at any suitable site of entry. Entry into the left ventricle 14 through the apex or apical region of the heart (e.g., at or adjacent to the apex 72) is illustrated by FIG. 5. Typically, access into the left ventricle 14, for instance, to perform a mitral valve repair, is gained through making a small incision into the apical region, close to (or slightly skewed toward the left of) the median axis 74 of the heart 10. Typically, access into the right ventricle 18, for instance, to perform a tricuspid valve repair, is gained through making a small incision into the apical region, close to or slightly skewed toward the right of the median axis 74 of the heart 10. The apex/apical region of the heart is a bottom region of the heart that is within the left or right ventricular region but is distal to the mitral valve 22 and tricuspid valve 26 and toward the tip or apex 72 of the heart 10. More specifically, an “apex region,” or “apical region,” of the heart is within a few centimeters to the right or to the left of the septum 20 of the heart 10. Accordingly, the left and right ventricles can be accessed directly via the apex 72, or via an off-apex location that is in the apical region, but slightly removed from the apex 72, such as via a lateral ventricular wall, a region between the apex and the base of a papillary muscle, or even directly at the base of a papillary muscle. The device 500 can access the heart in any manner.

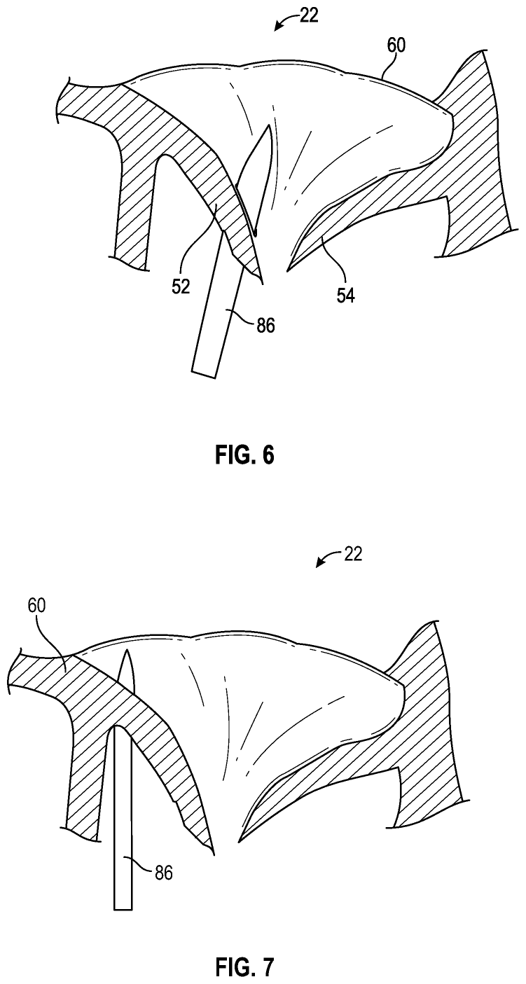

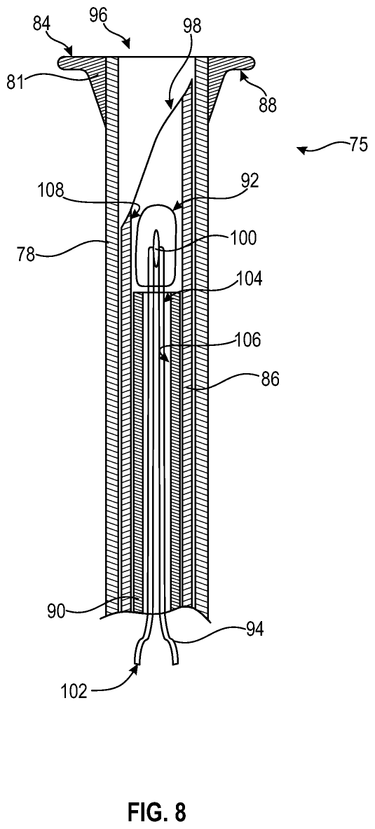

The access device 500 can be used to provide needles 86 (see FIGS. 6-22) and/or delivery devices 75 to access cardiac valves. Various procedures can be performed in accordance with the needles and delivery devices described herein in order to effectuate a cardiac valve repair, which will depend on the specific abnormality and the tissues involved. For example, needles 86 (see FIG. 6) and/or needle delivery devices 75 (see FIG. 8) can be used for a wide variety of different procedures, including, but not limited to, the implantation of one or more artificial chordae tendineae into one or more leaflets of a malfunctioning mitral valve 22 and/or tricuspid valve 26, an Alfieri procedure, and an annuloplasty procedure, and a wide variety of other procedures. In an exemplary embodiment, the needles are configured to pass through the native valve tissue, deploy a repair component, and retract back through the valve tissue. Referring to FIG. 6, in the case of an implantation of an artificial chorda, the needle 86 is inserted through the valve leaflet 52. As will be described in more detailed below, a repair device 92 (not shown in FIG. 6) is deployed. Then, the needle is retracted. The repair device 92 can be secured to another portion of heart tissue to complete the repair. FIG. 7 illustrates the use of a needle to perform an annuloplasty. The needle 86 is inserted through the valve annulus 60 as shown in FIG. 7, a repair device (not shown in FIGS. 7 and 8) is deployed, and the needle is retracted.

As illustrated in FIG. 5, the needle delivery devices 75 can be introduced into the ventricle 14 of the heart and advanced in such a manner so as to contact one or more cardiac tissues (for instance, a leaflet 52, 54, an annulus 60, a cord 42, a papillary muscle 43, or the like) that are in need of repair. Sonic guidance, for instance, TEE guidance or ICE, can optionally be used to assist in the advancement of the device into the ventricle.

The needle delivery devices 75 described herein can take a wide variety of different forms. Referring to FIGS. 8-11, in one exemplary embodiment, the needle delivery device 75 comprises a main shaft 78 with an interior lumen and a functional distal portion 81 having a tip 84 configured for repairing a cardiac valve tissue, for instance, a mitral valve leaflet 52, 54 or annulus 60. The tip 84 can take a wide variety of different forms. In the illustrated embodiment, the tip can have an atraumatic blunt end, to avoid pushing the entire device through the valve tissue, such as a leaflet 52, 54 or annulus 60. An end protector 88 can be provided at the distal end of the shaft 78 to provide the blunt end. In some embodiments, the end protector 88 can comprise an expandable balloon. In one exemplary embodiment, the distal portion 81 comprises a suction device for holding the tissue, such as leaflet tissue 52, 54 still as the tissue is acted upon by the needle 86. The suction device can take a wide variety of different forms. One exemplary suction device that can be used is disclosed in U.S. Patent Application Publication No. 2017/0304050 A1, which is incorporated herein by reference in its entirety. The needle delivery device 75 can be manipulated in such a manner so that a selected cardiac tissue (for instance, a papillary muscle, one or more leaflet tissues, chordae tendineae, or the like) is contacted with the functional distal portion 81 of the needle delivery device 75 and a repair effectuated, for instance, a mitral or tricuspid valve repair.

In the example illustrated by FIG. 8, a needle 86 having a distal end 98 and an interior lumen 108 is disposed in the delivery device 75. A repair device 92 and a repair device ejector 90 are disposed in the needle 86. The repair device 92 can take a wide variety of different forms. Examples of repair devices 92 include, but are not limited to, knots, pledgets, anchors, and the like. The repair device 92 can be any device or structure for providing a reinforcement or backing to tissue, such as leaflet tissue 52, 54 or annulus tissue 60. The repair device ejector 90 can take a wide variety of different forms. Any device capable of deploying the repair device 92 from the needle can be used. In the example illustrated by FIG. 8, the repair device ejector 90 comprises a hollow tube. In the example illustrated by FIG. 8, a suture 94 is connected to the repair device 92 and extends through the hollow ejector 90. The term “suture” is used herein according to its broad and ordinary meaning, and may refer to any elongate strip, strand, wire, tie, line, string, ribbon, strap, or other form or type of material that may be used in medical procedures. Although a single suture is described in some embodiments, it should be understood that such description is applicable to any number, form, or configuration of suture(s).

In certain embodiments, the repair device 92 comprises a pledget or other tissue anchor will. In some embodiments, the pledget 92 has suture tails 102, 94 running at least partially therethrough in a delivery configuration, as shown in FIGS. 8 through 11. Having a pledget or other type of tissue anchor with pre-attached suture tails in accordance with some implementations of the embodiments of FIGS. 8 through 11 can advantageously provide a relatively efficient deployments and anchoring process. For example, once the pledget or other tissue anchor 92 is deployed from the needle 86, there may advantageously be no need to further attach sutures or other tethers to the anchor, as sutures are pre-attached to the tissue anchor 92.

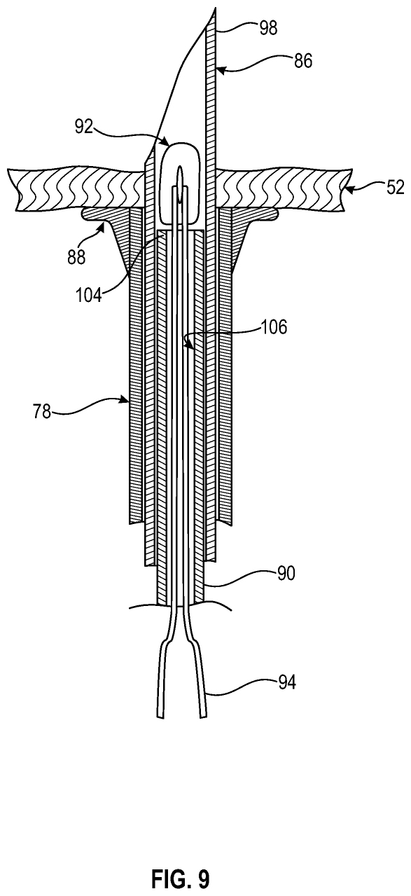

Referring to FIGS. 8 and 9, the needle 86 may be configured to be slidably disposed (e.g., slip-fit) within the main shaft 78. In FIG. 9, the distal end 98 of the needle 86 extends from the distal end 96 of the main shaft 78. The distal end 98 of the needle 86 may be configured to penetrate the target tissue 52, as shown. In some embodiments, the needle 86 can be electropolished to make it smooth. The repair device 92 has a stored position and a deployed position. The repair device 92 in stored in the distal end 98 of the needle 86. The repair device may be compressed to allow storing. The stored repair device 92 can be parallel with the axis of the needle 86, compressed or otherwise configured to allow for storage in a small area. The repair device 92 can be configured to expand and/or rotate when deployed out of the needle 86. In some embodiments, the deployed repair device 92 is substantially orthogonal to the axis of the needle 86.

The suture 94 can take a wide variety of different forms. For example, the suture 94 can be a suture, a wire, etc. In some embodiments, the suture 94 is a suture made of PTFE or ePTFE material. In some embodiments, the suture 94 comprises UHMwPE (ultra-high molecular weight polyethylene) material (e.g., DYNEEMA®, Koninklijke DSM, Heerlen, The Netherlands), for example, FORCE FIBER® suture (Teleflex Medical, Gurnee, Ill.). The distal end 100 of suture 94 is attached to the repair device 92. The proximal end 102 of suture 94 extends from the proximal end of the main shaft 78 and the ejector 90. In one embodiment, the suture 94 is looped through the center of the repair device 92. The loop 100 is formed at the distal end of the suture 94, leaving two proximal ends 102.

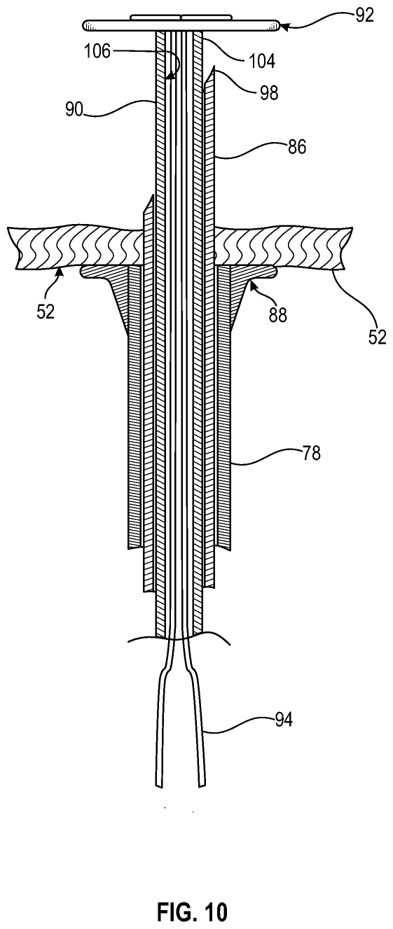

Referring to FIGS. 9 and 10, the ejector 90 is slidably disposed (e.g., slip-fit) within the needle 86. In FIG. 10, a distal portion 104 of the ejector 90 extends from the distal end 98 of the needle 86, for example on the atrium side of valve leaflet tissue 52. The ejector 90 is configured to push the repair device 92 out of the distal end 98 of the needle 86. In the embodiment illustrated, the ejector 90 comprises an interior lumen 106. The suture 94 passes through the interior lumen 106 of the ejector 90. The distal end 104 of the ejector 90 prevents the repair device 92 from entering into the interior lumen 106 of the pledget ejector 90. Alternatively, the suture 94 can pass through the interior lumen 108 of the needle 86 and run parallel with the pledget ejector 90.

FIGS. 9-11 show the operation of the needle delivery device 75. As illustrated in FIG. 9, the needle delivery device 75 has been positioned at a desired repair area. For example, the end projector 88 may be localized to the intended valve leaflet tissue 52, 54 or annulus tissue 60. The needle 86 may be advanced to puncture through the tissue. In some embodiments, both the ejector 90 and the suture 94 move with the needle. The repair device 92 remains inside the needle 86.

As illustrated in FIG. 10, the needle 86 can be maintained for a period in a puncture position. The ejector 90 advances and pushes the repair device 92 out of the needle 86. The repair device 92 may be moved to its deployed position and configuration. For example, the repair device 92 can move to a position that is orthogonal to the axis of the needle 86.

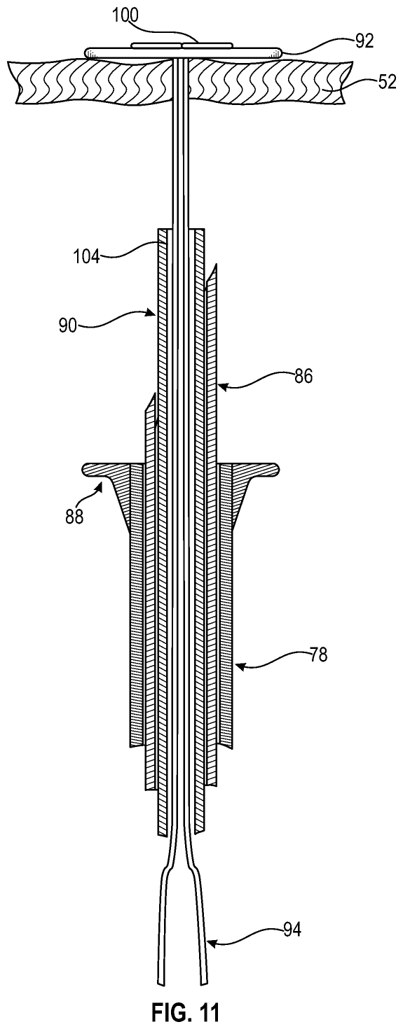

As illustrated in FIG. 11, the main shaft 78, the needle 86, and the ejector 90 are withdrawn from the tissue 52 while remaining in their extended condition of FIG. 11. In some alternative embodiments, the ejector 90 and/or the needle 86 can be retracted into the main shaft 78 before moving the main shaft 78. The repair device 92 with the loop 100 covers the tissue puncture site and can be used to anchor the suture 94 on to another tissue area to connect the repair device 92 to another repair device.

Another aspect of the present disclosure is an improved needle. In some embodiments, the needle is a hypodermic needle. The disclosed needles are designed to provide cardiac tissue penetration, such as valve leaflet tissue 52, 54 or valve annulus tissue 60, with reduced axial tensile force and/or a smaller cutting area. The reduced force and/or smaller cutting area prevents coring of the tissue. Preventing coring allows the puncture wound to seal itself immediately or more quickly when the needle is removed. Coring is the effect of needles forming a “crescent moon” shaped cut, followed by the displacement of the flap created by the cut. The problem with this coring cutting action is that the resulting cut hole can be large and thereby reduce the holding ability of the repair device through tissue, such as a valve leaflet and/or valve annulus tissue.

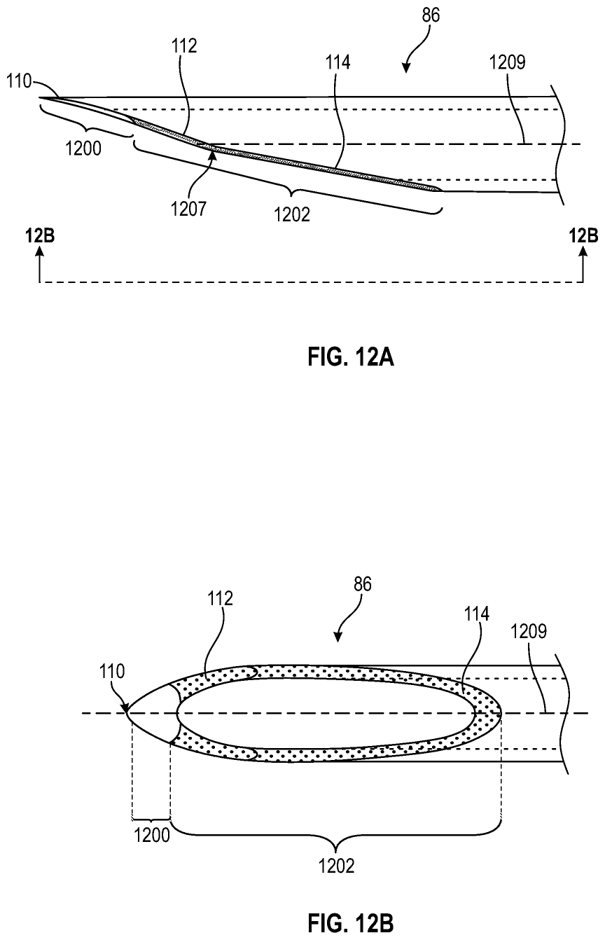

Referring to FIGS. 12A and 12B, in one exemplary embodiment, the configuration of the needle tip changes the way the needle penetrates through tissue and leaves a smaller cut area once the needle is removed. The needle 86 comprises a tip 110, a distal beveled edge 112, and a proximal beveled edge 114. The tip 110 can be very sharp or pointed to facilitate initial penetration of the needle through the tissue. A portion 1200 of the distal beveled edge 112 can have sharp cutting surfaces. A remainder of the distal beveled edges 112 and proximal beveled edges 114 have smoothed, non-cutting surfaces. The portion 1202 (e.g., the non-sharp surface of the beveled edge 112 and the beveled edge 112) can be made smoothed in a wide variety of different ways. For example, the portion 1202 can be polished in any conventional manner. In one exemplary embodiment, the portion 1202 is smooth, with non-cutting electropolished surfaces. The non-cutting surfaces are configured to stretch the tissue rather than cut it. Thus, non-cutting surfaces result in a reduced size puncture hole when the needle 86 is removed, that is the stretched tissue can return to its original size or close to its original size, where cut tissue cannot.

The distal beveled portion 112 may be formed from two beveled cuts, each deflecting away from the tip 110. Furthermore, the portion 1200 of the distal beveled portion 112 may advantageously be relatively sharp compared to the radiused edges/surfaces of the proximal beveled surface 114 and the radiused portion 1202 of the distal beveled surface/portion 112. The distal 112 and proximal 114 beveled portion may be separated by an inflection point 1207, which may be aligned with or near the central axis 1209 of the needle 86. The radiused portion 1202 of the needle may induce stretching of the tissue rather than cutting tissue. For example, the radiused portion 1202 may cause the needle puncture to be dilated during the insertion of the needle and deployment of the pledget, suture knot, memory metal wire anchor, or other type of distal anchor. When the needle is removed, the dilated tissue may relax back to its previous form to result in a relatively smaller puncture dimension.

With only the portion 1200 of the distal beveled portion 112 remaining un-radiused and relatively sharp, the tissue cut area may be relatively smaller than for needles having distal penetration portions that are not radiused. Furthermore, tissue stretch area may be maximized or relatively larger. The sharp portion 1200, however, may promote ease of tissue penetration when cutting through tissue during initial needle penetration.

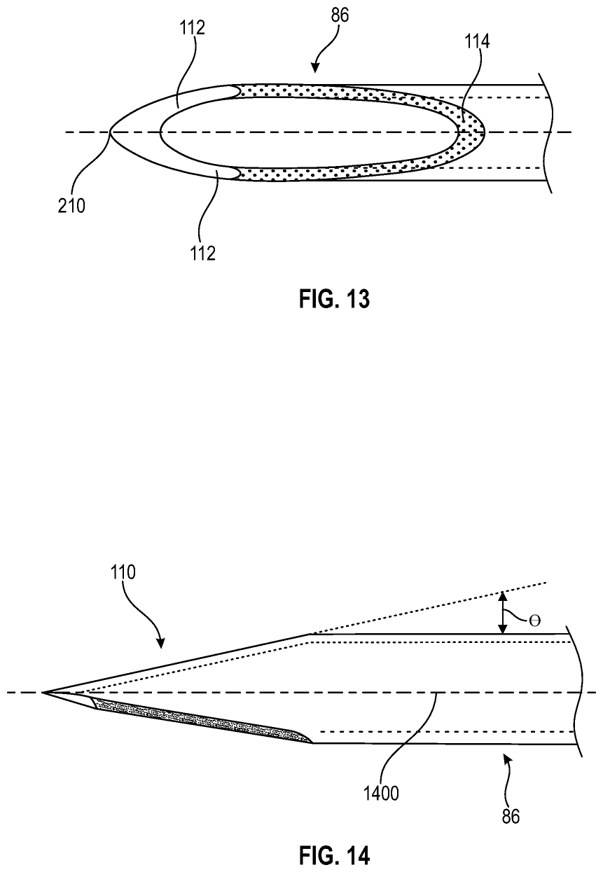

In other exemplary embodiments illustrated in FIG. 13, all beveled edges 112, 114 are radiused such that once a tissue penetration is achieved, the advancement of the needle 86 stretches the tissue rather than cutting. In some embodiments, all beveled edges 112, 114 are electropolished or polished to form a radius in some other manner. Radiused edges of bevels, away from the penetrating tip, can be from about 25 to about 500 μm (from about 0.001 to about 0.02 inch), or any sub range in between. In some exemplary embodiments, the radiused edges of bevels can be from about 130 to about 400 μm (from about 0.005 to about 0.015 inch), or any range in between. In some other exemplary embodiments, the radiused edges of bevels can be from about 180 to about 300 μm (from about 0.007 to about 0.012 inch), or any range in between. In some exemplary embodiments, the radiused edges of bevels can be about 250 μm (about 0.01 inch). In some embodiments, the cutting tip 210 can be also radiused from about 25 to about 130 μm (from about 0.001 to about 0.005 inch) without greatly increasing the force to penetrate tissue. In some exemplary embodiments, the cutting tip 210 can be radiused from about 25 to about 250 μm (from about 0.001 to about 0.01 inch), or any range in between. In some embodiments, the tip 210 is electropolished to radius the tip. Alternatively, the tip can be radiused in some other manner.

FIG. 14 illustrates another embodiment of a needle 86. An angle θ or bend can be provided near the sharp end of the needle. The bend is configured to cause a centralized initial tissue puncture (e.g., aligned with a central axis 1400 of the needle 86) followed by stretching of tissue rather than cutting action. Because the tip 110 is aligned with the central axis 1400, the bent tip 110 will cause the initial loading of the needle tip 110 to be on the central axis 1400 of the needle 86. This on-axis loading prevents lateral movement and/or torqueing of the needle 86 as it penetrates through tissue.

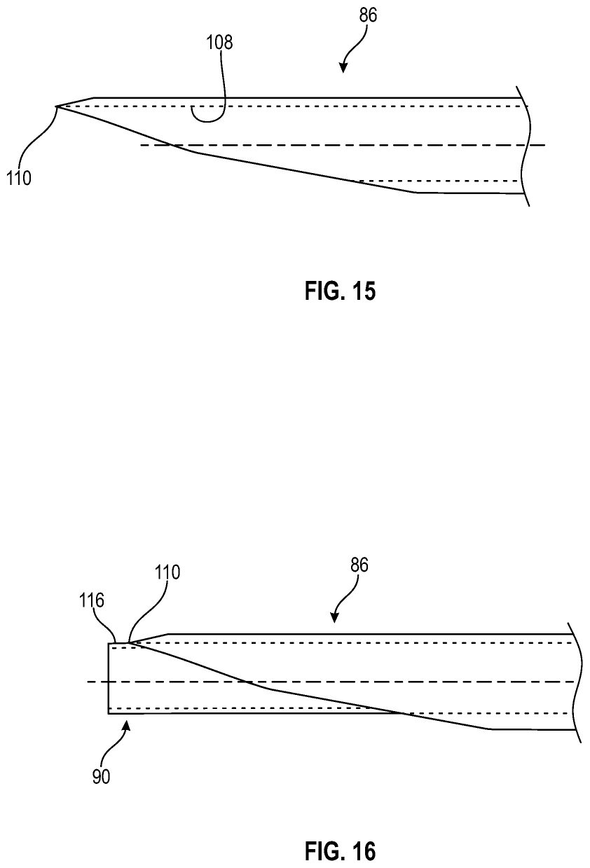

FIG. 15 illustrates another exemplary embodiment of a needle 86. In the example illustrated by FIG. 15, the sharp tip 110 of the needle 86 is coincident with the interior lumen wall 108 of the needle 86. Referring to FIG. 16, this positioning of the sharp tip 110 creates additional protection for the suture 94, both for initial deployment as well as for subsequent deployments. That is, the positions of the sharp tip 110 adjacent to the outside surface 116 of the ejector 90 prevents direct contact of the suture 94 with the sharp tip 110.

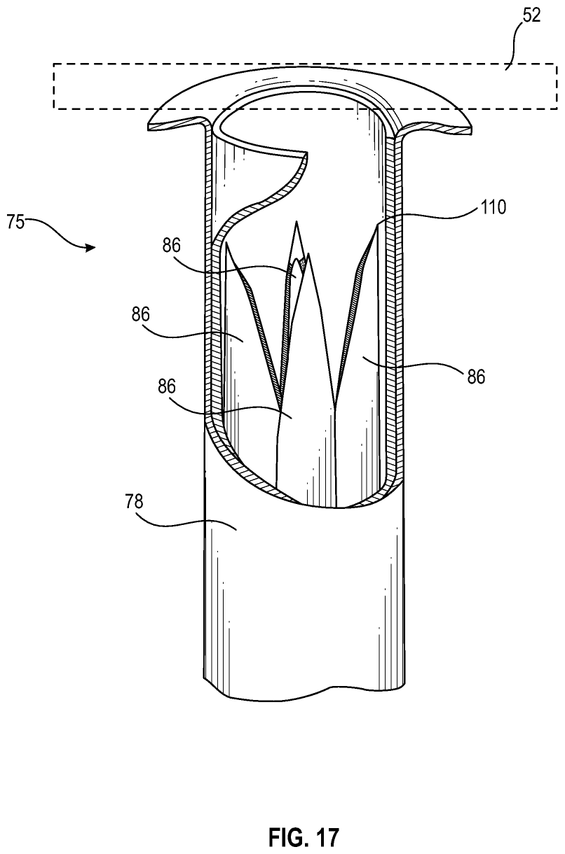

FIG. 17 illustrates another embodiment of the needle delivery device 75. The illustrated needle delivery device 75 contains more than one needle 86, which allows for the placement of multiple repair devices 92, thus reducing the number of penetrations needed through the valve introducer. The needle delivery device can contain 2, 3, or more needles with pre-loaded repair devices and/or lines 94. The number of needles can be selected to optimize the volume of the main shaft 78 of the delivery device 75 that is filled with needles. For example, three, or five needles can result in a majority of a circular tube being filled with the needles.

In certain embodiments, the repair device 92 comprises a pledget or other tissue anchor will. In some embodiments, the pledget 92 has suture tails (e.g., suture tail(s) 94) running at least partially therethrough in a delivery configuration, as shown in FIGS. 17 through 22. Having a pledget or other type of tissue anchor with pre-attached suture tails in accordance with some implementations of the embodiments of FIGS. 17 through 22 can advantageously provide for relatively efficient deployment and/or anchoring processes. For example, once the pledget or other tissue anchor 92 is deployed from the needle 86, there may advantageously be no need to further attach sutures or other tethers to the anchor, as sutures are pre-attached to the tissue anchor 92.

Referring to FIG. 17, prior to deployment, the sharp tips 110 of the needles 86 are rotated to be against the inside wall of the main shaft 78. This protects the sharp tip 110 from damage and prevents the sharp tip 110 from damaging a suture 94 or lines which have been previously deployed.

During initial deployment as illustrated in FIG. 18, the needle 86 can be rotated for example 180 degrees, causing the sharp tip 110 to be located near the center of the main shaft 78. Positioning the sharp tip 110 near the center of the main shaft promotes easier placement and improved accuracy of the repair device 92 placement. In other exemplary embodiments, the needle tips 110 are not positioned against the wall 78 and/or the needles are not rotated for deployment.

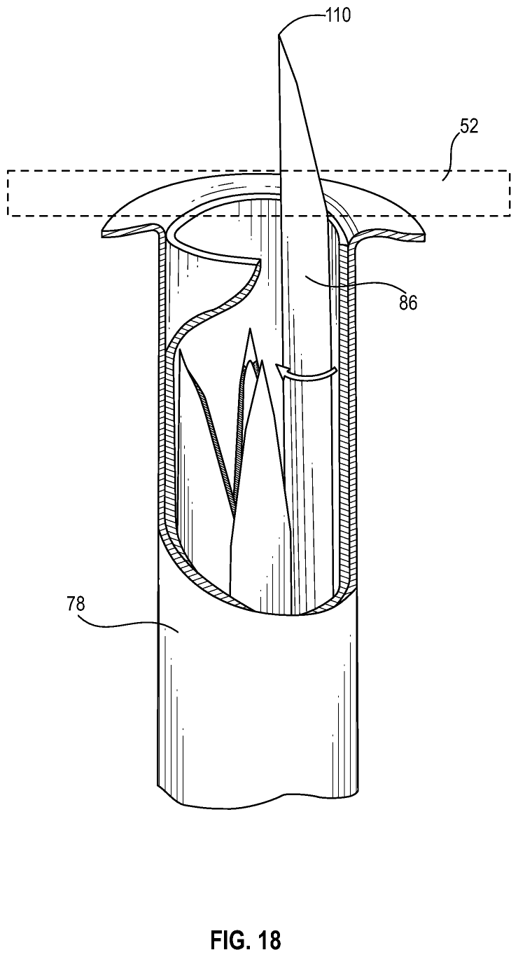

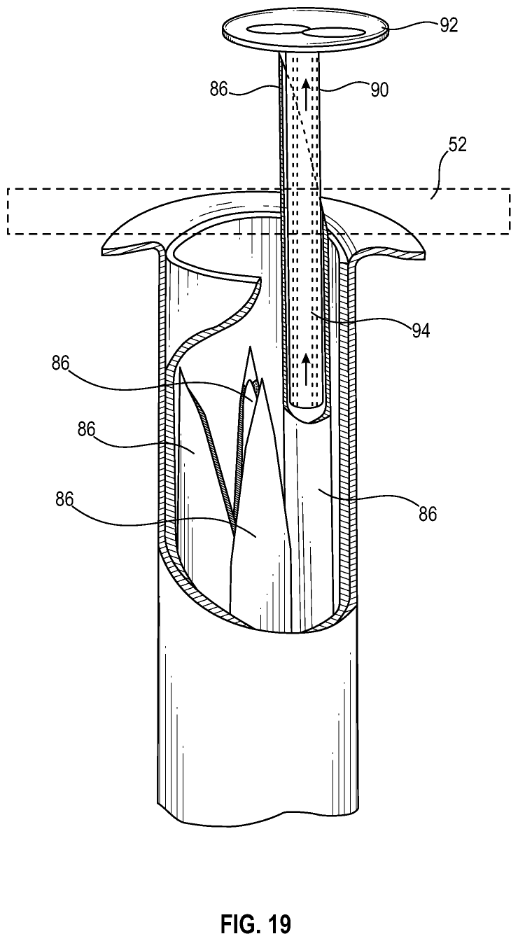

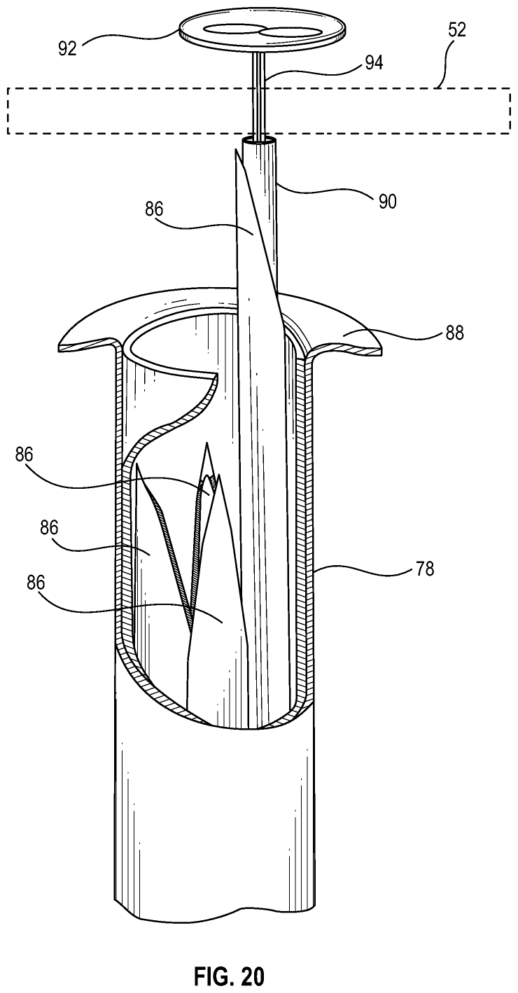

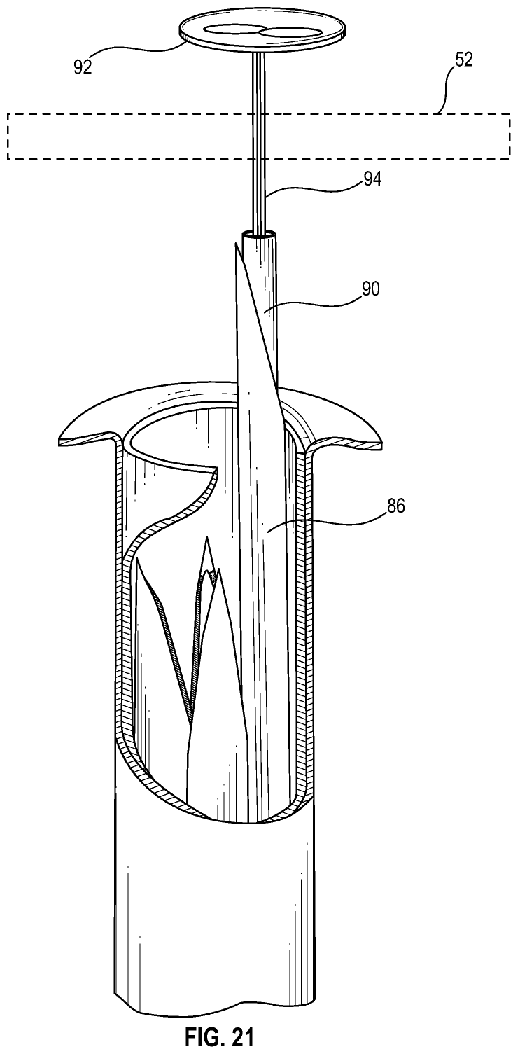

Once the needle is fully displaced as illustrated in FIG. 19, the ejector 90 deploys the repair device 92 and the suture 94 out of the needle 86. The repair device 92 is deployed in an orthogonal position with respect to the penetrated tissue prior to pull-back of the needle 86. FIGS. 20 and 21 illustrate retraction of the needle 86 and the ejector 90. The retraction of the needle 86 and the ejector 90 can be done in the same manner as described with respect to FIGS. 10 and 11. Once the ejector 90 is deployed, it can optionally remain in the extended position in order to protect the suture 94 and prevent damage to other lines which can have previously been deployed as illustrated in FIGS. 20 and 21.

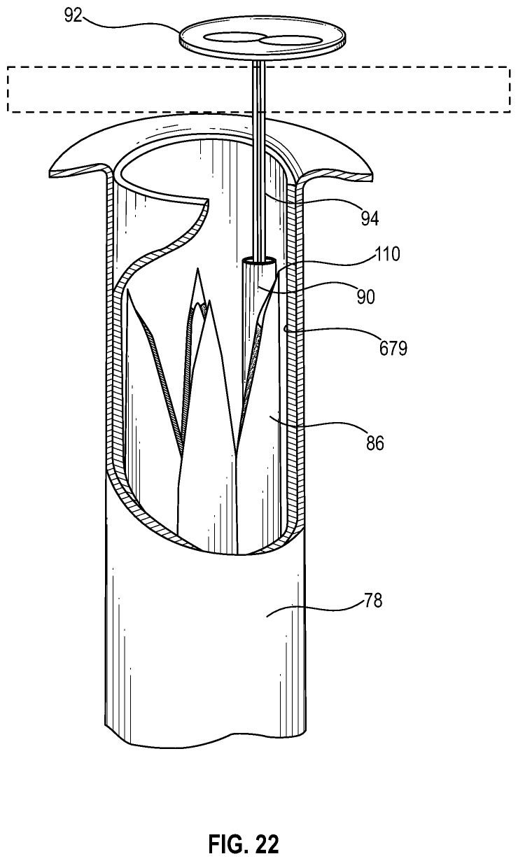

Once the needle is pulled back out of the penetrated tissue as illustrated in FIG. 22, the needle 86 can optionally rotate 180 degrees, such that the sharp needle tip 110, once fully retracted, will again be positioned in such a way that the sharp tip 110 will be located against the inside wall 679 of the main shaft 78. Once the deployment process of the repair device 92 is complete, subsequent deployments can be accomplished with the same delivery device 75. The rotation of the needles 86, the configuration of the needles, and/or radiusing of edges of the needles reduces the possibility of damaging a previously deployed line.

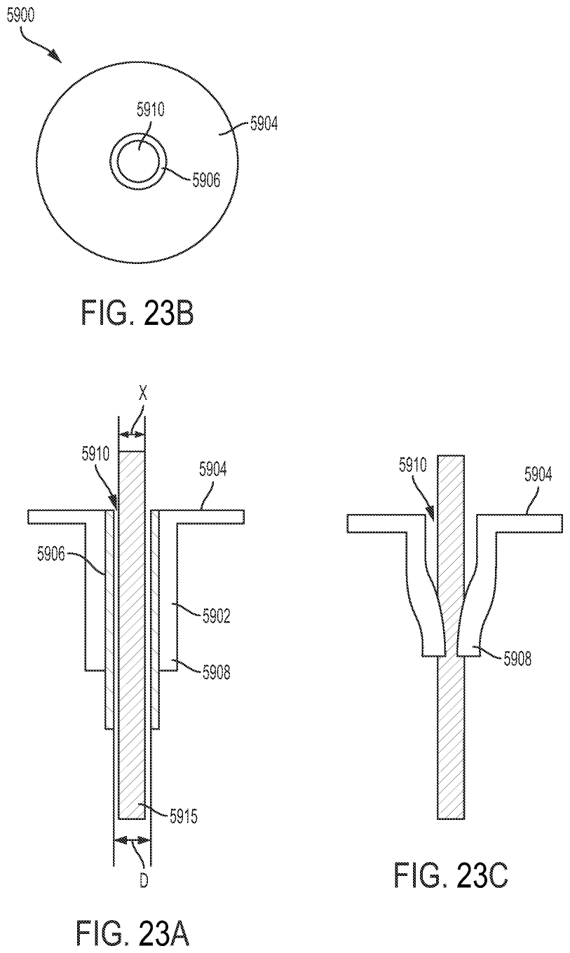

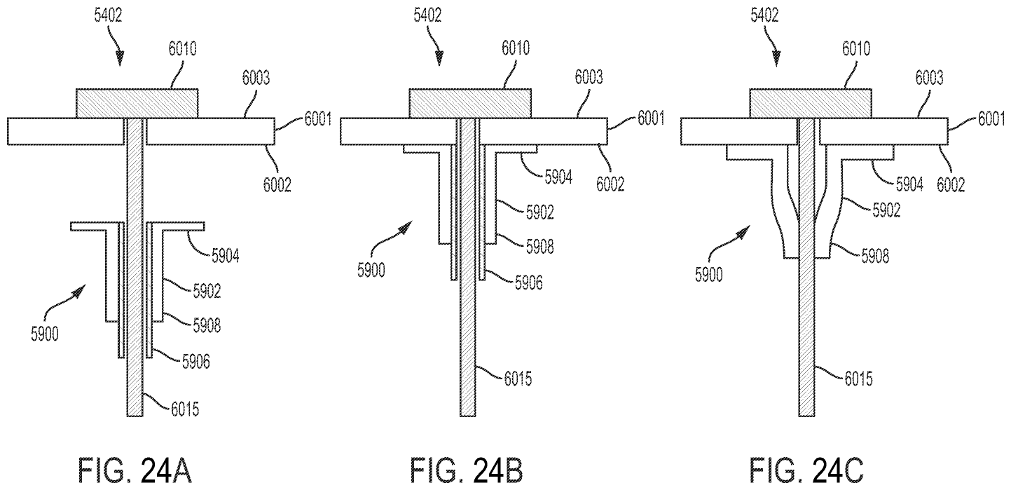

Referring to FIGS. 23A-23B and 24A-24C, an exemplary embodiment of an anchor member 5900 includes a compression portion 5902, an abutment portion 5904, a placement member 5906, and an opening 5910 that extends through the abutment portion 5904 and the compression portion 5902. The opening 5910 is configured to receive a suture portion 5915 of an attachment member, such as at least a portion of the suture 94 described above. The compression portion 5902 is configured to compress the suture portion 5906 of an attachment member to prevent the attachment member from moving. The placement member 5906 is configured to expand the compression portion 5902 so that the opening 5910 has a larger diameter D than the diameter X of the suture portion 5915. The placement member 5906 allows the anchor member 5900 to be moved along the suture portion 5915 so that the anchor member 5900 can be placed in a desired location on the suture portion 5915. Once the anchor member 5900 is placed in a desired location, the placement member 5906 can be removed from the anchor member 5900, which causes at least a portion of the compression member 5902 to compress such that the diameter D of at least a portion of the opening 5910 is less than the diameter X of the suture portion 5915. That is, the compression portion 5902 is made of an elastic material or a shape memory material, such as, for example, plastic, steel, shape memory alloy material, such as Nitinol, any combination of these materials, and the like.

The compression portion 5902 is made so that it has an original shape (e.g., the shape of the compression portion 5902 that is shown in FIG. 23C). The original shape of the compression portion 5902 makes at least a portion of the opening 5910 have a smaller diameter D than the diameter X of the suture portion 5915. The placement member 5906 is configured to expand the compression portion 5902 such that the entire opening 5910 has a diameter D that is larger than the diameter X of the suture portion 5915, which allows the anchor member 5900 to be moved up and down the suture portion 5915. For example, the placement member 5906 may be a cylindrical sleeve. Upon removing the placement member 5906, the compression portion 5902 moves back to its original shape, which causes at least a portion of the compression portion 5902 to compress and secure the suture portion 5915 in a desired location. Referring to FIG. 23C, in the illustrated embodiment, the anchor member 5900 is configured such that a lower portion 5908 of the compression portion 5902 is configured to compress the suture portion 5915. In alternative embodiments, any other portion of the compression portion may be used to compress the suture portion 5915, or the entire compression portion 5902 can compress the suture or suture 5915. The abutment portion 5904 is configured to abut against an object that the attachment member is attached to, such as, for example, the annulus, an annuloplasty band, or any other object that the attachment member is attached to. The anchor member 5900 can be made of, for example, plastic, metal, steel, shape memory alloys, combinations of these materials, and the like. The placement member 5906 can be removed by holding the anchor in place and pulling the placement member 5906 or holding the placement member in place and advancing the anchor. In an embodiment, the anchor comprises a knot made from suture, for example, ePTFE suture that features a low-profile insertion configuration and a higher profile anchoring configuration.

FIGS. 24A-24C illustrate the exemplary anchor member 5900 attaching an exemplary embodiment of an attachment member 5402 to a tissue member 6001 (e.g., the annulus of the mitral valve, the annulus of the tricuspid valve, etc.). In the illustrated embodiment, the attachment member 5402 is a T-shaped attachment member that includes a securing portion 6010 and a suture portion 6015. The securing portion 6010 abuts a second side 6003 of the tissue member 6001, and the suture portion extends through the tissue member 6001 to a first side 6002 of the tissue member 6001. After the attachment member 5402 is attached to the tissue member 6001, the attachment member 5402 is secured to the tissue member 6001 by the anchor member 5900. Referring to FIG. 24A, the anchor member 5900 is moved along the suture portion 6015 with the placement member 5906 maintaining the opening 5910 in an expanded state. Referring to FIG. 24B, the anchor member 5900 is placed in a desired location in which the abutment portion 5904 of the anchor member 5900 is abutting the first side 6002 of the tissue member 6001. Referring to FIG. 24C, after the anchor member 5900 is placed in a desired location, the placement member 5906 is removed, which causes a lower portion 5908 of the compression portion to compress against the suture portion 6015. The compression by the compression portion 5902 on the suture portion 6015 secures the attachment member 5402 to the tissue member 6001.

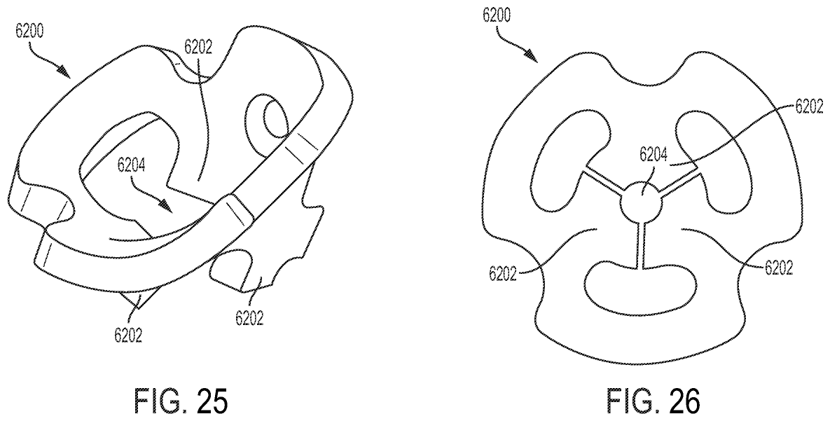

Referring to FIGS. 25 and 26, another exemplary embodiment of an anchor member 6200 includes three or more flap members 6202. The illustrated embodiment shows an anchor member 6220 that has three flap members 6202. In alternative embodiments, the anchor member 6200 can have four flap members, five flap members, etc. The flap members 6202 are deflectable, such that each of the flap members 6202 can move from an open position (FIG. 25) to a closed position (FIG. 26). For example, a set shape of the flap members can be the closed position and the flap members can be held in the open position by a placement member. When the placement member is removed, the flap members can spring back toward the closed position from the open position. In some embodiments, the anchor 6200 includes a protector component (not shown), which may protect the suture from damage. For example, certain suture types may be prone to damage in certain situations, such as ePTFE (e.g., GORE-TEX® CV5 and CV4 suture), polypropylene (e.g., Ethicon PROLENE® suture), and/or the like. The protector component may be placed inside the anchor in the pre-deployed state, such that the protector is compressed around the suture, thereby reducing exterior damage.

An optional opening 6204 is provided at a center location between the flap members 6202. When one or more of the flap members 6202 are in the open position, the opening 6204 is configured such that the anchor member 6200 can be moved along a suture portion of an attachment member. When all of the flap members 6202 are in the closed position, the opening 6204 is configured to compress the suture portion of an attachment member such that the suture portion is constrained in a radial direction. The anchor member 6200 is deployed in the open position and moved to a desired location on a suture portion of an attachment member. Once the anchor member 6200 is in the desired position, the flap members 6202 are simultaneously moved from the open position to the closed position. The flap members 6202 provide a force in the radial direction to secure the attachment member to a tissue member (e.g., to secure the attachment member to the annulus of the mitral valve). Alternatively, the flap members 6202 can be moved from the open position to the closed position in a sequential order or random order. During this alternative procedure, a tortuous path is crated with multiple holding points on the suture portion of the attachment member, which will increase the holding force on suture portions that have higher surface lubricities. The holding forces applied by the anchor member 6202, in effect, create a tourniquet around the suture portion. The anchor can be made from a wide variety of different materials. For example, the anchor member 6202 can be made from plastic, metal, such as steel, shape memory alloys, such as Nitinol, and/or any combination of these materials, and the like.

In certain embodiments, the anchor member 6200 can be used with a protecting member (not shown) that is used to prevent surface damage to a suture portion of an attachment member as it is being held by the anchor member 6200. The anchor member can be deployed by any suitable device, such as, for example, any of the valve repair devices disclosed in the present application.

The anchor members 5900, 6200, as well as any other anchor members described in the present application, can be used to secure any of the attachment members described in the present application, and can be used in any of the procedures described in the present application. The anchor members 5900, 6200 can also be used in a wide variety of additional procedures. For example, the anchor members 5900, 6200 can be used in any procedure that involves approximating a tissue member.

A person skilled in the art should readily understand that, the above disclosed embodiments can be implemented with each other. For example, an exemplary needle delivery device can comprise four needles with the improvements disclosed above and in FIGS. 12A, 12B, 14, and 15.

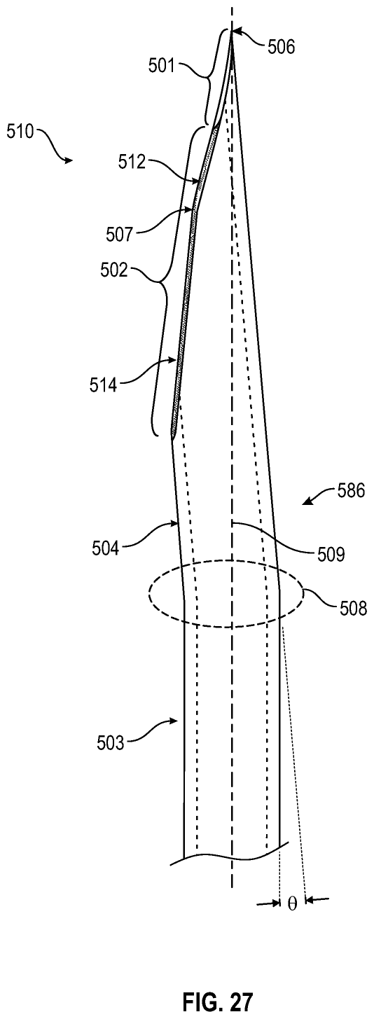

FIG. 27 illustrates an embodiment of a needle 586. A bend having an angle θ is implemented in the needle 586 at a position 508 along a length of the needle shaft. The area 508 may advantageously be approximately 1 cm or less from the tissue-piercing point 506 of the needle. The bend in the needle shaft can be configured to cause a centralized initial tissue puncture, followed by stretching of tissue rather than cutting due at least in part to a beveled edge associated with the portion 502 of the needle tip. For example, the angle θ may advantageously be implemented to cause the point 506 of the needle 586 to be aligned with a central axis 509 of the needle shaft 503. In some embodiments, the angle θ of the bend 508 is between approximately 3°-5°. Because the point 506 is aligned with the central axis 509, the bent needle tip may cause the initial loading of the needle tip 510 to be generally on the central axis 509 of the needle shaft 503. This on-axis loading can prevent or impede lateral movement and/or torqueing of the needle 586 as it penetrates through biological tissue or other material.

The tip 510 of the needle 586 represents a multiple-bevel needle tip, as described in detail herein. The multiple-bevel tip 510 may be formed using a plurality of bevel cuts. For example, a first bevel cut may be used to form the proximal beveled surface 514 at the base of the needle tip, which extends from the base of the needle tip 510 to the inflection point 507. In some implementations, a single bevel cut is used to form the proximal beveled surface 514, which may have the same surface plane on both sides of the point 506 of the needle tip 510.

The needle tip 510 may further be formed using one or more additional bevel cuts associated with the distal portion 512 of the needle tip 510 that is distal to the inflection point 507. For example, a first angled bevel cut may be made on a first side of the point 506 of the needle tip, whereas a second angled bevel cut may be made on the opposite side of the needle point 506 in the distal portion 512 of the needle tip 510. Such angled bevel cuts may advantageously form a relatively sharp tip portion 501 at or near the point 506 of the needle tip 510.

A radiused, or relatively blunt, edge may be created at the portion 502 of the needle tip 510, as described in detail herein. For example, the portion 502 of the needle tip 510 may be converted to a relatively non-sharp, or rounded, surface in order to induce dilation of the tissue at the puncture site. Generally, dulling of needle surfaces may be considered undesirable in some applications as increasing the resistance of puncture for the needle. In certain medical applications, such additional resistance may increase pain or discomfort associated with needle puncture. However, with respect to solutions relating to embodiments of the present disclosure, such additional puncture resistance may be acceptable as a trade-off for the induced dilation benefits described herein.

The bend 508 in the needle shaft may be an axial bend in the needle shaft designed to align the sharp point 506 of the needle tip 510 with the central axis 509 of the needle 586. The bend 508 in the needle shaft may advantageously minimize or reduce lateral movement of the needle during insertion of the tip 510 through biological tissue. In particular, with respect to certain other needles, the needle tip may tend to be deflected due to the angle of the needle tip surface(s). Such surface(s) may undesirably push or direct the needle tip away from the target penetration point, which may result in tissue damage or injury. With the point 506 of the needle tip 510 aligned with the center axis 509, a more centered position may be achieved, such that migration of the needle as it penetrates the target tissue may be reduced or prevented. Due to the bend 508, loading of the needle 586 may be generally coincident with the axis 509 of the needle shaft in one or both directions. In some implementations, when manufacturing the needle 586, the bend 508 may be implemented prior to implementing the bevel cuts associated with the needle tip 510.

The radiused portion 502 of the needle tip surface may be implemented in any suitable or desirable way. For example, needle tip radiused edge may be achieved using electropolishing or other similar technology. For example, when radiusing the portion 502 of the needle tip surface, the portion 501 that is desired to remain relatively sharp may be covered such that it is not exposed to the electrochemical processes for radiusing the portion 502, such that only a relatively small portion 501 of the needle tip remaining relatively sharp. When puncturing biological tissue, the through-hole produced may be relatively small for the needle 586, wherein the tissue around the through-hole may be inclined to dilate, rather than tear, thereby advantageously producing a relatively small through-hole. When the needle tip 510 is subsequently withdrawn from the puncture site, the punctured tissue may be inclined to recede, such that only the small puncture hole produced by the relatively sharp portion 501 of the needle tip 510 remains. Although the illustration of FIG. 27 shows an inflection bend 508, in some embodiments, the alignment of the point 506 of the needle tip 510 with the central axis 509 is achieved through a curved or continuous bend.

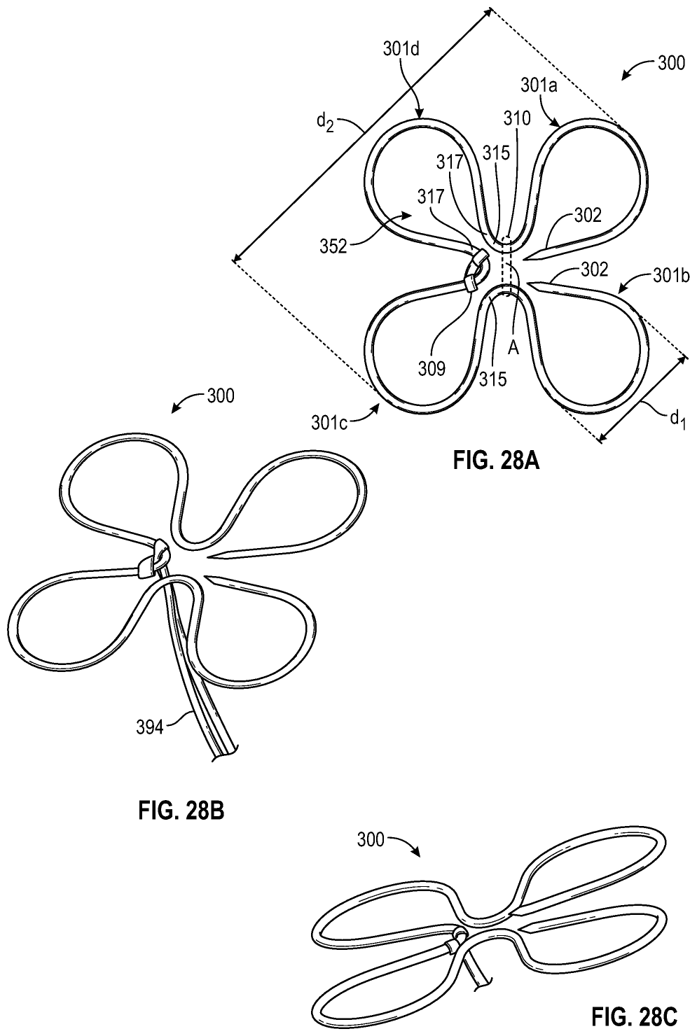

In some implementations, the present disclosure relates to pre-shaped tissue anchors configured to provide increased or desirable holding force when deployed on biological tissue, such as on the distal/atrial side of a heart valve leaflet, as described in detail herein. FIGS. 28A-28C illustrate views of a pre-shaped wire tissue anchor 300 in accordance with one or more embodiments of the present disclosure. The anchor 300 may be used in accordance with certain embodiments disclosed herein as an alternative to certain knot-type anchors illustrated and described herein. In its deployed configuration, as illustrated in FIGS. 28A-28C, the anchor 300 may have a generally planar body/form including a plurality of coplanar force-distributing arms or projections 301 that may extend generally radially outward from a center of the anchor 300. Although the tissue anchor 300 is illustrated as having four projections/arms 301, tissue anchors in accordance with the present disclosure can have greater or less than four projections/arms. Furthermore, although projections/arms are illustrated and described, it should be understood that the force-distributing features 301 may have any form or shape.

In some embodiments, the projections/arms 301 are formed of a single wire or form, as shown. Although the illustrated anchor 300 is formed to have two free ends 302, the ends of the anchor may be joined or integrated in some manner. The projections/arms 301 may advantageously be shaped and/or configured such that the anchoring force exerted by the projections/arms is relatively evenly distributed over and against the tissue surface (e.g., atrial side of valve leaflet) when the device is deployed. The projections/arms 301 can advantageously be angularly/circumferentially spaced apart from one another to a maximum degree. The anchor 300 may have a relatively thin and flat profile, which may reduce the risk of thrombus in some implementations.

The anchor 300 can be self-expandable and can be formed from a shape-memory material, such as Nitinol, such that the anchor 300 self-expands from a delivery configuration to a deployed configuration when released or deployed from a delivery system or apparatus. In some embodiments, the anchor 300 is formed at least in part from a plastically-expandable material, such as stainless steel or cobalt-chromium alloy, and can be configured to be plastically expanded from a delivery configuration to a deployed configuration by an expansion device. In some embodiments, the anchor 300 may be laser cut or otherwise formed from a flat sheet of metal, such as Nitinol. Alternatively, the anchor 300 can be formed by bending one or more metal wires into the form shown.

The projections/arms 301 can extend perpendicularly or substantially perpendicularly to a central axis A of the anchor 300, which generally may align with tethered suture(s) 394 that are coupled to the anchor in some manner. The suture(s) 394 may be tied or attached to the anchor 300 in any suitable or desirable manner. Although the suture 394 is shown as looped/tied around a connection portion between two projections/arms, suture(s) may be coupled to the anchor at other locations, including across an inner diameter of the anchor 300, such as is shown by the dashed-line suture 310 in FIG. 38A.

Each of the projections/arms 301 can comprise a respective loop-shaped member spaced including an open area 352 therein. Each projection/arm 301 may include two circumferentially-spaced radial inner ends 317 that are connected to adjacent radial inner ends of one or more adjacent projections/arms by respective connecting portions 315. The projections/arms 301 and the connecting portions 315 of the anchor 301 may collectively form a simple open- or closed-loop structure wherein a single continuous frame member forms each of the projections/arms and the connecting portions.

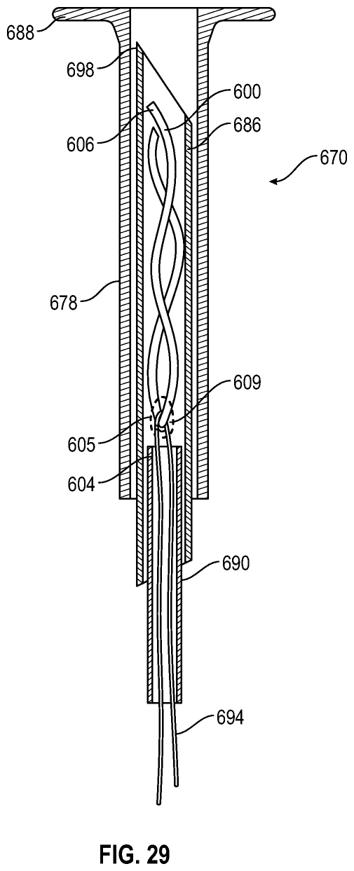

As shown in FIG. 28A, the anchor 300 in the deployed configuration can include a projection/arm diameter d₁ and an outer diameter d₂. The outer diameter d₂ can be defined by the diameter formed from the radial outermost ends of the projections/arms. The number of arms, the length of the arms, and the diameter dimensions of the anchor 300 can be varied as needed for particular anchoring applications. Compared to certain other tissue anchors, the anchor 300 may advantageously provide comparable or greater anchoring/retention force using less material/metal, and therefore may be less susceptible to thrombus formation, be relatively easier to deliver and deploy, and/or provide other benefits. Although a single anchor 600 and associated suture(s) are shown in the needle 686 in FIG. 29, in certain embodiments, multiple pre-shaped tissue anchors may be delivered and/or contained within the needle 686.

FIGS. 29-31 illustrate a tissue anchor 600 and associated delivery system 670 at various stages of a tissue anchor deployment process in accordance with one or more embodiments. Embodiments of pre-shaped tissue anchors in accordance with the present disclosure may be implemented at least in part by shape-setting an anchor profile in a metal (e.g., memory metal alloy, such as Nitinol) that is relatively elastic (e.g., highly-elastic or super-elastic). In some embodiments, such tissue anchors may comprise material(s) and/or form(s) that allow for the shape of the tissue anchor to be at least partially straightened or manipulated into a pre-deployed state or configuration that allows for relatively easy delivery and placement of the shape-set anchor form as an anchor for attached suture(s).

Referring to FIGS. 29-31, a needle 686 may be configured to be slidably disposed (e.g., slip-fit) within a shaft or lumen 678 of the delivery system 670. The anchor 600 can be compressed, straightened, or otherwise constricted to a delivery configuration for delivery to the target anatomy in the delivery system 670. In the delivery configuration, the anchor 600 can be placed and retained in a generally straightened/compressed configuration in which projections/arms thereof are elongated to form one or more lengths of relatively straightened wire/material. Although the wire 600 is shown in a folded delivery configuration, in some embodiments, the anchor 600 is not folded or overlapped in the delivery configuration. In the delivery configuration, the suture(s) 694 may advantageously be pre-attached/coupled to the anchor 600, as shown. For example, as in the illustrated embodiment, the suture(s) 694 may be attached to the anchor 600 at or near a fold 605 in the anchor. In some embodiments, the suture(s) 694 may be attached to the anchor 600 at a crimped wire end portion thereof.

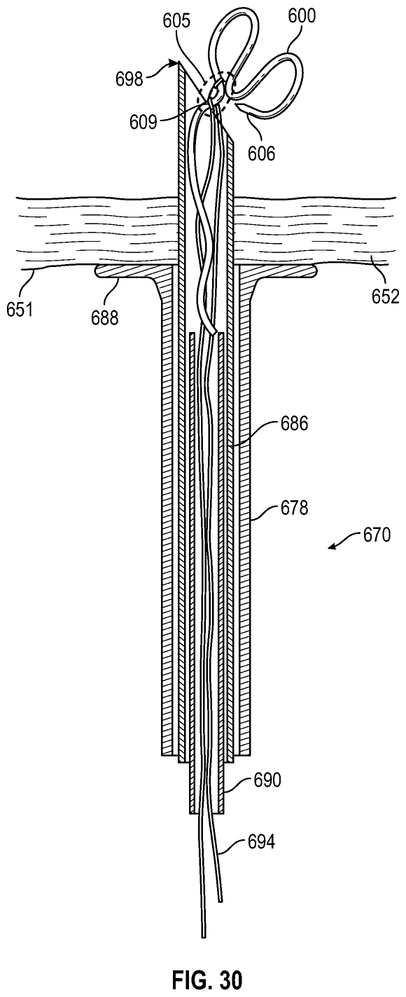

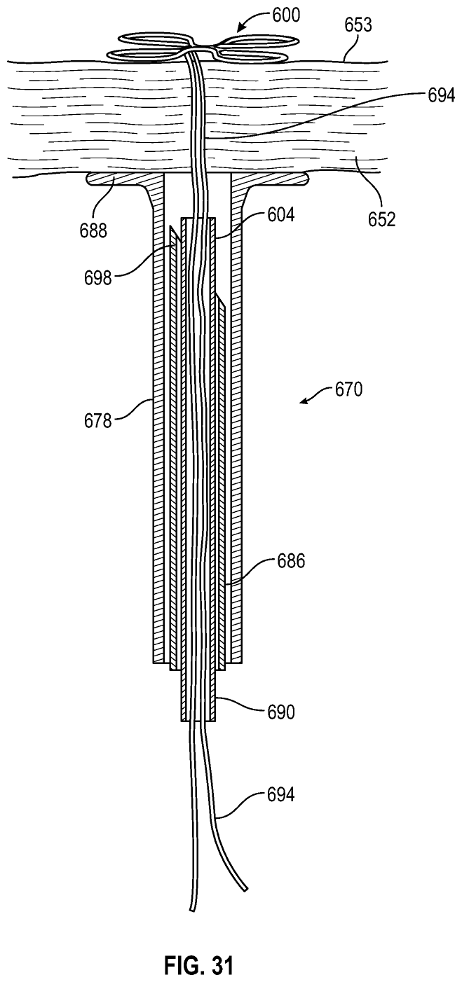

In FIG. 30, the distal end 698 of the needle 686 has been extended from the distal end of the main shaft 678. The distal end 698 of the needle 686 may be configured to penetrate the target tissue 652, as shown. In some embodiments, at least a portion of the tip portion of the needle 686 can be electropolished to make it smooth to promote tissue dilation rather than cutting. The anchor 600 may be configured to assume a delivery configuration, as shown in FIG. 29, as well as a deployed configuration, as shown in FIG. 31, wherein FIG. 30 shows a partially-deployed configuration.

In some embodiments, the tissue anchor 600 in stored in a distal end portion of the needle 686. With reference to FIG. 29, the tissue anchor 600 may be compressed/straightened to allow for storing in the delivery system 670, as shown. The stored tissue anchor 600 can generally be placed in a configuration in which one or more portions of the wire anchor are at least partially parallel with the longitudinal axis of the needle 686, such as in a compressed/straightened state or otherwise configured to allow for storage in a relatively small area. With reference to FIG. 30, the tissue anchor 600 can be configured to expand and/or rotate when deployed out of the needle 686. The projections/arms thereof can be heat-set or otherwise shaped to extend axially away from each other when deployed.

The tissue anchor 600 may advantageously be coupled to one or more sutures 694 at portion 605. That is, the portion 605 of the anchor wire 600 may comprise a suture-attachment feature, such as a fold or bend in the wire, an eyelet, a hook, a clamp or crimp in the wire, or the like. In some embodiments, the suture(s) 694 may be tied to the anchor 600 or otherwise engaged with the suture-attachment feature 605. The suture(s) 694 can take a wide variety of different forms. For example, the suture 694 can be a suture, a wire, a tape, a band, a string, or the like. In some embodiments, the suture 694 comprises PTFE or ePTFE material. In some embodiments, the suture 694 comprises UHMwPE (ultra-high molecular weight polyethylene) material (e.g., DYNEEMA®, Koninklijke DSM, Heerlen, The Netherlands), for example, FORCE FIBER® suture (Teleflex Medical, Gurnee, Ill.). The proximal ends of the suture tails 694 may extend from the proximal end of the delivery system shaft 678 and/or a pusher/ejector 690 component of the delivery system 670.

The pusher/ejector 690 can be slidably disposed (e.g., slip-fit) within the needle lumen. When deploying the tissue anchor 600, a distal portion 604 of the ejector 690 may extend from the distal end 698 of the needle 686, for example on the atrium side of valve leaflet tissue. The pusher/ejector 90 may be configured to push the tissue anchor 600 out of the distal end 698 of the needle 686. In the embodiment illustrated, the pusher/ejector 90 comprises an interior lumen through which the suture tails 694 may be passed. In some embodiments, the distal end 604 of the pusher/ejector 690 prevents the tissue anchor 600 from entering into the interior lumen of the pusher/ejector 90. Alternatively, the suture 694 may pass through the interior lumen of the needle 686 and run parallel with the pusher/ejector 690.

With reference to FIGS. 30 and 31, the delivery system 670 may be positioned against a surface 651 of target biological tissue 652. For example, an atraumatic tip 688 of the delivery system 670 may be localized to the ventricle side of a target valve leaflet in some implementations. The atraumatic tip 688 may comprise silicone or other at least partially flexible material in a flange configuration at a distal end of the main shaft 678. The needle 686 may be advanced to puncture through the tissue 652. In some embodiments, both the pusher/ejector 690 and the suture 694 move with the needle. The tissue anchor 600 may remain inside the needle 686 until it is ejected therefrom using the pusher/ejector 690. The pusher/ejector 90 may be advanced to push the tissue anchor 600 out of the needle 686.

As shown in FIG. 31, the tissue anchor 600 may be moved to its deployed position and configuration as it is ejected from the needle 686. For example, the tissue anchor 600 can move or be drawn to a position that is substantially orthogonal to the axis of the needle 686 for placement against the distal surface 653 of the target tissue 652. In some embodiments, the tissue anchor wire is configured to assume the deployment configuration in response to a stimulation of some type, such as an electrical or thermal stimulation. Once the tissue anchor 600 is deployed, the delivery system 670, the needle 686, and the pusher/ejector 690 may be withdrawn from the tissue 652. As deployed, the tissue anchor 600 and suture(s) 694 may cover the tissue puncture site and can be used to anchor the suture 694 onto another tissue area to connect the tissue anchor 600 to another tissue anchor. In some embodiments, the anchor 600 is deployed in combination with a pledget form, which may provide desirable force-distribution and/or tissue protection.

The anchor 600 may be used to anchor artificial chordae tendineae to heart valve leaflets, as described in detail herein, and/or may be used for other types of tissue-approximation therapies. Pre-shaped wire tissue anchors in accordance with embodiments of the present disclosure can provide for relatively easy placement of the tissue anchors on the distal side of tissues along with attached sutures. In some embodiments, the anchor 600 may be delivered and/or deployed having a mesh/cloth covering or sleeve around at least a portion thereof, which may serve to protect the adjacent biological tissue and/or provide other benefits. In some embodiments, the anchor is coated or wrapped in material designed and/or configured to promote in-growth of tissue therewith, which may help prevent against tissue abrasion over time. Once deployed, the shape-set wire anchor 600 can assumes its pre-shaped form, thereby providing increased resistance against pulling through the puncture hole in the tissue. The anchor 600 may comprise a wire form that is advantageously rigid enough to be maintained on a distal side of the target tissue without the propensity to be drawn back through a puncture hole implemented to deploy the anchor 600. Furthermore, the anchor 600 may advantageously be sufficiently rigid to prevent or reduce irritation or my corporation against the adjacent tissue.

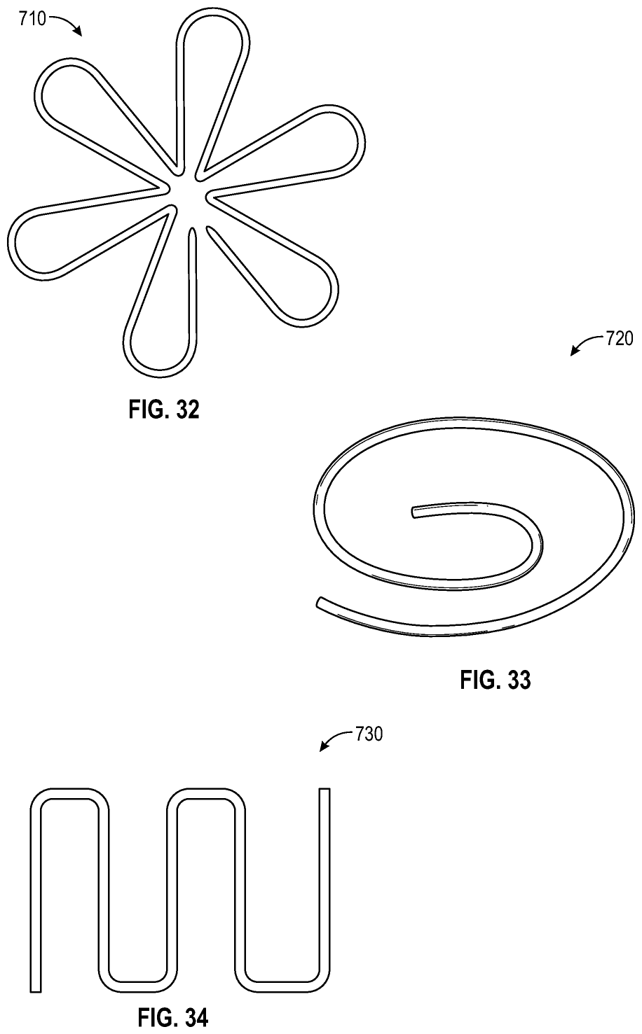

Although the tissue anchors in FIGS. 28-31 are illustrated as having a clover-type shape with loop-shaped projections/arms, it should be understood that anchors and/or projections/arms thereof can have a variety of shapes. For example, shapes for projections/arms may generally have one or more narrow portions and one or more wide portions, which may be configured to desirably distribute anchoring forces. Some embodiments of projections/arms or features for tissue anchors in accordance with the present disclosure have any desirable shape including, but not limited to, a mushroom shape, a diamond shape, a circular in shape, or any other shape. In some embodiments, the configuration of one or more of the projections/arms can be different from one or more other projections/arms of the anchor. In some embodiments, the projections/arms of a tissue anchor need not comprise loop-shaped members with central openings and instead can comprise elongated wires or strut members that are secured to a common central portion at only one end of the wire or strut member. FIGS. 32-34 illustrate additional example tissue anchor forms in accordance with embodiments of the present disclosure, including a flower-type anchor 710 comprising petal-type projections, a spiral-type anchor 720, and a grill-type anchor, respectively.

It is contemplated that the devices and methods disclosed herein can be used in procedures outside the heart. That is, while the embodiments have been described with reference to a heart valve, the needles, devices and methods described above can be used in any procedure that requires penetrating a tissue and providing a reinforcement on the far side thereof. In view of the many possible embodiments to which the principles of the disclosed invention can be applied, it should be recognized that the illustrated embodiments are only preferred examples of the invention and should not be taken as limiting the scope of the invention. All combinations or sub-combinations of features of the foregoing exemplary embodiments are contemplated by this application. The scope of the invention is defined by the following claims. We therefore claim as our invention all that comes within the scope and spirit of these claims.

What is claimed is:

1. A tissue anchor comprising:
a memory metal wire configured to transition between an at least partially straightened delivery configuration and an expanded deployed configuration forming an anchor; and
a suture-attachment feature configured to have a suture coupled thereto;
wherein, in the expanded deployed configuration, one or more portions of the memory metal wire extend radially outward from a center of the tissue anchor.

2. The tissue anchor of claim 1, wherein, in the expanded deployed configuration, the memory metal wire forms a plurality of loop projections.

3. The tissue anchor of claim 2, wherein the suture-attachment feature comprises a connection portion of the memory metal wire between circumferentially-spaced radial inner ends of adjacent loop projections of the plurality of loop projections.

4. The tissue anchor of claim 1, further comprising a suture coupled to the suture-attachment feature and having two suture tails extending therefrom.

5. The tissue anchor of claim 1, wherein, in the expanded deployed configuration, the memory metal wire forms a clover form.

6. The tissue anchor of claim 5, wherein the clover form has two free ends.

7. The tissue anchor of claim 1, wherein, in the expanded deployed configuration, the memory metal wire is flat.

8. The tissue anchor of claim 1, wherein the memory metal wire is configured to transition to the expanded deployed configuration in response to a stimulus.

9. The tissue anchor of claim 1, wherein, in the expanded deployed configuration, the memory metal wire forms a spiral form.

10. The tissue anchor of claim 1, wherein, in the expanded deployed configuration, the memory metal wire forms a grill form.

11. An anchor delivery system comprising:
a main shaft having an atraumatic tip and an interior lumen;
a needle having a distal end and an interior lumen, the needle being disposed within the interior lumen of the main shaft and configured to be extended from the distal end of the main shaft in a deployed position of the needle;
a pusher disposed within the interior lumen of the needle and configured to be extended from the distal end of the needle in a deployed position of the pusher;
a memory metal wire disposed in the interior lumen of the needle in an at least partially straightened delivery configuration, the memory metal wire being configured to automatically assume an expanded deployed configuration when ejected from the interior lumen of the needle by the pusher; and
a suture coupled to a suture-attachment feature of the memory metal wire within the interior lumen of the needle.

12. The anchor delivery system of claim 11, wherein the memory metal wire is in a folded configuration.

13. The anchor delivery system of claim 12, wherein the suture-attachment feature is a bend in the memory metal wire.

14. An anchor delivery system comprising:
a main shaft having an atraumatic tip and an interior lumen;
a needle having a distal end and an interior lumen, the needle being disposed within the interior lumen of the main shaft and configured to be extended from the distal end of the main shaft;
a pusher disposed within the interior lumen of the needle and configured to be extended from the distal end of the needle;
a tissue anchor disposed in the interior lumen of the needle in an at least partially compressed delivery configuration, the tissue anchor being configured to automatically assume an expanded deployed configuration when ejected from the interior lumen of the needle by the pusher; and
one or more suture tails attached to the tissue anchor and disposed within the interior lumen of the needle.

15. The anchor delivery system of claim 14, wherein the tissue anchor is a pledget.

16. A method of deploying a tissue anchor, the method comprising:
providing an anchor delivery system comprising:
a main shaft having an atraumatic tip and an interior lumen;
a needle having a distal end and an interior lumen, the needle being disposed within the interior lumen of the main shaft and configured to be extended from the distal end of the main shaft in a deployed position of the needle;
a pusher disposed within the interior lumen of the needle and configured to be extended from the distal end of the needle in a deployed position of the pusher;
a memory metal wire disposed in the interior lumen of the needle in an at least partially straightened delivery configuration, the memory metal wire being configured to automatically assume an expanded deployed configuration when ejected from the interior lumen of the needle by the pusher; and
a suture coupled to a suture-attachment feature of the memory metal wire within the interior lumen of the needle;
positioning the atraumatic tip of the main shaft against a target tissue;
moving the needle to the deployed position, thereby puncturing through the target tissue with the needle;
ejecting the memory metal wire from the interior lumen of the needle while the needle is in the deployed position; and
forming a memory metal wire into an expanded tissue anchor form on a distal side of the target tissue.

17. The method of claim 16, further comprising:
pre-shaping the memory metal wire in the expanded tissue anchor form;
compressing the memory metal wire into a compressed delivery configuration; and
inserting the memory metal wire into the interior lumen of the needle in the compressed delivery configuration.

18. The method of claim 16, wherein the expanded tissue anchor form has a clover shape comprising a plurality of radially-extending loop projections.

19. The method of claim 16, wherein said moving the needle to the deployed position comprises puncturing the target tissue with a point of the needle being substantially aligned with a longitudinal axis of the main shaft.

20. The method of claim 19, wherein the needle comprises an elongated shaft forming the interior lumen of the needle, the elongated shaft having a bend feature that is configured to align the point of the needle with a longitudinal axis of the elongated shaft.
